(19) United States
(12) Patent Application Publication
(10) Pub. No.: US 2025/0171825 A1
(43) Pub. Date: May 29, 2025

(54) METHODS FOR PROCESSING AND BARCODING NUCLEIC ACIDS

(71) Applicant: VILNIUS UNIVERSITY, Vilnius (LT)

(72) Inventors: Linas MAZUTIS, Vilnius (LT); Greta LEONAVICIENE, Vilnius (LT); Karolis GODA, Vilnius (LT)

(21) Appl. No.: 18/715,668

(22) PCT Filed: Dec. 1, 2022
(86) PCT No.: PCT/EP2022/084074

§ 371 Date: May 31, 2024

Related U.S. Application Data
(60) Provisional application No. 63/284,669, filed on Dec. 1, 2021.

(51) Int. Cl. C12Q 1/6806; C12Q 1/686

(52) U.S. Cl. CPC C12Q 1/6806; C12Q 1/686

(57) ABSTRACT

The present invention concerns a method comprising co-encapsulating a microcapsule and a particle in a droplet, the microcapsule comprising a semi-permeable shell and a core, wherein the core comprises a nucleic acid for processing, and wherein the particle comprises a reagent for use in the processing of the nucleic acid.

Part of this invention was made with funding supported under Grant No. 01.2.2-MITA-K-702-12-0003 awarded by the Agency for Science, Innovation and Technology (Lithuania).

### FIELD OF THE INVENTION

The present invention generally relates to methods of nucleic acid processing and analysis, and in particular to processing and analysis of the nucleic acid comprised in a biological species, such as a cell. Some aspects of the method involve co-encapsulation of (i) a semi-permeable microcapsule carrying nucleic acid; and (ii) a particle carrying a molecular tag, into a droplet, and attaching the molecular tag to the nucleic acid within the droplet. In particular, the method can be utilized for barcoding nucleic acid molecules from a plurality of cells. In one example, the method comprises isolating individual biological species (e.g., cells, bacteria, etc.) in semi-permeable microcapsules (SPMs), lysing the cells to release their internal content including nucleic acid molecules within the SPM, co-encapsulation of the SPMs carrying a cell lysate including nucleic acid molecules along with hydrogel beads containing DNA barcoding oligonucleotides into a microfluidic droplet (along with any necessary assay reagents) and labeling nucleic acids molecules from lysed cells with barcoded oligonucleotides within a microfluidic droplet. The specific sequence (barcode) within barcoding DNA oligonucleotides can be used to distinguish the barcoded nucleic acid molecules of one cell lysate from those of another cell lysate even when the nucleic acid molecules are pooled together. After sequencing the barcodes may be used to distinguish from tens to millions of nucleic acids arising from different cells or other biological species. The invention also relates to a plurality of co-encapsulated microcapsules and particles, and kits for performing the methods described herein.

### BACKGROUND OF THE INVENTION

Single-cell sequencing technologies offer new opportunities to delineate the intricate differences between individual cells and reveal biological mechanisms that are not discernible in bulk studies [1-12]. Over the last few years, droplet microfluidics has been established as an enabling technology for barcoding and sequencing single-cells [13]. In general terms the basic principle of the state-of-the-art droplet-based technologies can be summarized as follows: a mixture of cells is encapsulated into microfluidic droplets together with barcoded oligonucleotide primers (attached to hydrogel beads), reverse transcription (RT) and lysis reagent mix (FIG. 1). The mRNA released from the lysed cells remains trapped inside the same droplet and is tagged (barcoded) with oligonucleotide primers during RT reaction. After barcoding step, the material from all droplets is released and pooled by breaking the droplets, and the copy DNA (cDNA) library is processed for next-generation sequencing. Because each droplet carries primers encoding only one, unique barcode, which is different from barcodes in other droplets, the nucleic acids (such as mRNA) of individual cells are being labelled with a unique sequence tag. After sequencing the tags and barcodes can be demultiplexed allowing the reconstruction the cell population structure and single-cell resolution. The beads carrying barcoded oligonucleotide primers are typically synthesized by a combinatorial synthesis [13]. The barcode in this context encodes two parts: a cellular barcode, which indicates a cell from which mRNA is captured, and a unique molecular identifier (UMI), which provides a quantitative measure of absolute transcript levels in a given cell. The barcoded oligonucleotide primers besides a barcode sequence may also comprise nucleic acid capture sequence (e.g., poly(dT), gene specific sequence), PCR adapter and/or sequencing adapter, and may also contain other functional sequences (e.g., T7 promoter). The barcoded oligonucleotide primer may be single-stranded or double-stranded.

However, all state-of-the-art droplet-based microfluidics assays [13-17] suffer from technical and conceptual limitations. Once the cells are isolated inside the water-in-oil droplets, removing or replacing the reagents inside the compartments becomes a challenging task. Although some microfluidic tools such as droplet fusion or injection can facilitate new reagents introduction into pre-formed droplets at defined time points [18-23], such operations are still cumbersome to implement and require high level of expertise. Therefore, single-cell genomic assays in droplets are largely built on biochemical reaction conditions that are compatible with RT or PCR reaction, and that do not compromise droplet stability [24-26]. Performing multi-step or higher order biochemical reactions is challenging due to incompatible requirements of different enzymes, inhibitory salt effects, inability to replace buffers, remove unused reagents or add new ones. As a result, performing single-cell nucleic acid barcoding inside the droplets relies on mild lysis conditions that leads to suboptimal reaction yields during RT and PCR reactions. To improve the cDNA reaction yields it is desirable to lyse cells efficiently, however, harsh lysis reagents such as SDS, or guanidinium chloride are incompatible with RT and PCR assays. Removing inhibitory compounds from droplets having encapsulated cells from water-in-oil droplets is practically impossible to achieve.

### SUMMARY OF THE INVENTION

In a first aspect the present invention provides a method comprising: co-encapsulating a microcapsule and a particle in a droplet, the microcapsule comprising a semi-permeable shell and a core, wherein the core comprises a nucleic acid for processing, and wherein the particle comprises a reagent for use in the processing of the nucleic acid.

In a second aspect the present invention provides a method comprising: co-encapsulating a plurality of microcapsules and a plurality of particles in a plurality of droplets, each particle of the plurality of particles comprises a molecular tag, and each microcapsules of the plurality of microcapsules comprises a core, a semi-permeable shell, and a nucleic acid obtained from a biological species, wherein the nucleic acid is comprised in the core, and wherein:
(i) the plurality of droplets comprises one or more of the plurality of microcapsules and one of the plurality of particles;
(ii) the molecular tag of the particle in one droplet is distinguishable from the molecular tags of the particles in the other droplets,
   the method optionally comprising (a) releasing the molecular tag from the particle and/or releasing the nucleic acid from the microcapsule; and (b) attaching the molecular tag to the nucleic acid in the droplet.

In a third aspect the present invention provides a plurality of droplets produced by the method of the first or second aspects of the invention.

In a fourth aspect the present invention provides a droplet comprising a microcapsule and a particle, the particle comprises at least one molecular tag, and the microcapsule comprising a core, a semi-permeable shell, and a biological species comprised in the core.

In a fifth aspect the present invention provides a droplet comprising a microcapsule and a particle, the particle comprises at least one molecular tag, and the microcapsule comprising a core, a semi-permeable shell, and a cell lysate comprised in the core, wherein the cell lysate comprises cellular RNA and/or DNA.

In a sixth aspect the present invention provides a composition comprising a plurality of droplets according to the fourth or fifth aspects of the invention, each droplet comprising a particle with a different molecular tag and a microcapsule with a different biological species or cell lysate comprised in the core, wherein the cell lysate comprises cellular RNA and/or DNA.

In a seventh aspect the present invention provides a method for barcoding nucleic acid, comprising:
encapsulating a plurality of cells in a plurality of semi-permeable microcapsules such that the majority of the semi-permeable microcapsules contain no more than one cell;
lysing the encapsulated cells in order to release a lysate comprising nucleic acids within the plurality of semi-permeable microcapsules;
treating the plurality of semi-permeable microcapsules comprising the lysates in order to purify the released nucleic acids and/or deplete one or more enzymatic inhibitors present in the lysates;
producing a plurality of microfluidic droplets using a microfluidic device, the plurality of microfluidic droplets comprising the plurality of semi-permeable microcapsules, a plurality of particles comprising molecular tags, and one or more assay reagents, wherein at least 1%, preferably at least 10%, more preferably at least 50%, of said plurality of droplets comprises a single microcapsule and a single particle, and one or more assay reagents;
labelling the nucleic acids with the molecular tags in the plurality of droplets to produce barcoded (tagged) nucleic acids, where the nucleic acids from each cell comprise the same cell barcode which is different from the cell barcode of nucleic acids from other cells in other droplets; and
releasing the barcoded nucleic acids by breaking the plurality of droplets.

In an eight aspect the present invention provides a method, for barcoding nucleic acid, comprising:
encapsulating a plurality of cells in semi-permeable microcapsules such that the majority of the semi-permeable microcapsules contain no more than one cell;
lysing the encapsulated cells in order to release a lysate comprising the nucleic acids within the semi-permeable microcapsules;
treating the semi-permeable microcapsules comprising the lysates in order to purify the released nucleic acids and/or deplete one or more enzymatic inhibitors present in the lysates;
producing a plurality of microfluidic droplets each containing, on average, more than one semi-permeable microcapsule, a single particle comprising a molecular tag, and one or more assay reagents, using a microfluidics device;
labelling the nucleic acids with molecular tags in the plurality of droplets to produce barcoded (tagged) nucleic acids, where the nucleic acid in each droplet comprises the same cell barcode which is different from the cell barcode of the nucleic acids in other droplets.
releasing the barcoded nucleic acid molecules by breaking the plurality of droplets.
   In a ninth aspect, the present invention provides a method for amplifying and barcoding nucleic acid, comprising:
encapsulating a plurality of cells in a plurality of semi-permeable microcapsules such that the majority of microcapsules contain no more than one cell,
lysing the encapsulated cells in order to release a lysate comprising nucleic acids within the plurality of semi-permeable microcapsules,
treating the plurality of semi-permeable microcapsules comprising the lysate in order to purify the released nucleic acids and/or deplete one or more enzymatic inhibitors present in the lysates;
amplifying the nucleic acids in the microcapsules using a DNA polymerase driven reaction;
producing a plurality of microfluidic droplets comprising a plurality of the semi-permeable microcapsules, a plurality of particles comprising a molecular tag and one or more assay reagents, wherein at least 1%, preferably at least 10%, more preferably at least 50%, of said plurality of microfluidic droplets comprise a single microcapsule and a single particle, and one or more assay reagents;
labelling the amplified nucleic acids with the molecular tag in the plurality of droplets to produce barcoded (tagged) nucleic acids, where the nucleic acids from each droplet comprise the same cell barcode which is different from the cell barcode of nucleic acids from other cells in other droplets; and
releasing the barcoded nucleic acid molecules by breaking the plurality of droplets.
   In a tenth aspect, the present invention provides method for barcoding cDNA, comprising:
encapsulating a plurality of cells in a plurality of semi-permeable microcapsules such that the majority of semi-permeable microcapsules contain no more than one cell,
lysing the encapsulated cells in order to release a lysate comprising nucleic acids within the plurality of microcapsules;
treating the plurality of semi-permeable microcapsules comprising the lysates in order to purify the released nucleic acids and/or deplete one or more enzymatic inhibitors present in the lysates;
converting the released RNA to cDNA in the semi-permeable microcapsule using a reverse transcriptase reaction;
producing a plurality of microfluidic droplets comprising a plurality of the semi-permeable microcapsules, a plurality of particles comprising a barcoding DNA oligonucleotide and one or more assay reagents, wherein at least 1%, preferably at least 10%, more preferably at least 50%, of said plurality of droplets comprise a single microcapsule and a single particle, and one or more assay reagents;
labelling the cDNA with barcoding DNA oligonucleotides in the plurality of droplets to produce barcoded nucleic acids, where the cDNA from each droplet comprises the same cell barcode, which is different from the cell barcode of cDNA from other cells in other droplets; and
releasing the barcoded nucleic acid comprising cDNA by breaking the plurality of droplets.
   In an eleventh aspect the present invention provides a method for barcoding fragmented nucleic acids, comprising:
encapsulating a plurality of cells in a plurality of semi-permeable microcapsules such that majority of microcapsules contain no more than one cell,
lysing the encapsulated cells in order to release a lysate comprising nucleic acids within the plurality of semi-permeable microcapsules,
treating the plurality of semi-permeable microcapsules comprising the lysates in order to purify the released nucleic acids and/or deplete one or more enzymatic inhibitors present in the lysates;
fragmenting the released nucleic acids in the microcapsules using chemical, enzymatic or physical means;
optionally: amplifying the fragmented nucleic acids using DNA polymerase;
producing a plurality of microfluidic droplets comprising a plurality of the semi-permeable microcapsules, a plurality of particles comprising a molecular tag and one or more assay reagents, wherein at least 1%, preferably at least 10%, more preferably at least 50%, of said droplets comprise a single microcapsule and a single particle, and one or more assay reagents;
labelling the fragmented and optionally amplified nucleic acids with the molecular tags in the plurality of droplets to produce barcoded nucleic acids, where the nucleic acids from each droplet comprise the same cell barcode which is different from the cell barcode of nucleic acids from other cells in other droplets; and
releasing the barcoded nucleic acid molecules by breaking the plurality of droplets.
   Further embodiments of the invention are set out in the dependent claims and described below.

The invention revealed here makes use of a microcapsule comprising a semi-permeable shell and a core to carry nucleic acid obtained from a biological species into a droplet. In particular embodiments relating to nucleic acid obtained from cells, the methods may involve lysing individual cells within microcapsules comprising a semi-permeable shell and a core (also referred to herein as “semi-permeable microcapsules” or “(SPMs)”) and barcoding the nucleic acids of the cell lysate. In contrast to state-of-the-art droplet microfluidics systems, the cell lysis can be performed under harsh conditions (e.g. using guanidinium chloride, SDS, treating with DNAse I, etc) before subjecting the cell lysate to barcoding reaction. Upon cell lysis most of the nucleic acid molecules may be retained inside the SPM. As a result, the encapsulated cell lysate can be processed through multi-step sequential procedures to clean-up or otherwise treat the lysate, for example, by dispersing SPMs in aqueous buffer containing chaotropic agent and then removing chaotropic agent by dispersing SPMs in another aqueous buffer, before the cell lysate (in the SPM) is co-encapsulated with the particle carrying the barcode into the droplet for barcoding. Improved cell lysis, nucleic acids clean-up and removal of inhibitory compounds from a resulting cell lysate are critical for ensuring efficient nucleic acid barcoding reaction. Essentially, the nucleic acids of the cell lysate may be cleaned, purified or otherwise treated to improve the subsequent enzymatic and/or chemical reactions on nucleic acid molecules. Furthermore, the nucleic acid molecules in a cell lysate can be modified, fragmented or processed through different enzymatic and biochemical treatments before initiating the barcoding reaction. This possibility provides additional advantage over existing methodologies. The SPMs suspended in aqueous buffer can be packed (concentrated) such that their delivery into droplets becomes ordered and synchronized, ensuring that the majority of droplets host desirable number of SPMs, for example, exactly one SPM. As a result, the delivery of SPMs, along with hydrogel beads and assay reagents, to the same droplet can be precisely controlled.

The present invention generally relates to labelling of nucleic acids of cells using microfluidics. Further specific embodiments and aspects are as follows:

In one aspect, the present invention comprises the encapsulation of individual biological species (e.g., cells) in the plurality of liquid droplets that are then converted semi-permeable microcapsules (SPMs), while retaining the encapsulated cells. Although the cells are a main source of nucleic acid material, the nucleic acid may be introduced into the droplets from other sources, such as bacteria, viruses or microorganisms.

In another aspect, the present invention comprises a plurality of SPMs containing encapsulated cells being dispersed in an aqueous buffer to initiate, modify or terminate a desirable enzymatic or chemical reaction.

In another aspect, the present invention comprises the cells being lysed by dispersing the SPMs carrying cells in an aqueous solution containing lysis reagents.

In another aspect, the present invention comprises a plurality of SPMs containing single-cell lysates including nucleic acids being dispersed in an aqueous buffer to initiate, modify or terminate a desirable reaction.

In another aspect, the SPMs containing single-cell lysates are dispersed in an aqueous solution to replace the lysis reagents with other reaction components and salts.

In one aspect, part of cellular material (e.g. proteins, lipids, metabolites) may passively diffuse out from the interior part of the SPMs when suspended in an aqueous buffer. In another aspect, the nuclei acid molecules in a cell lysate that are longer than approximately ˜200 nt. may be retained within the SPMs.

In some cases, the encapsulated cells are being treated with deoxyribonuclease (DNAse) or ribonuclease (RNAse) in order to digest a selected type of nucleic acids. For example, the use of DNAse enzyme may hydrolyze DNA, but not RNA. And the use of RNAse enzyme will hydrolyze RNA, but not DNA.

In one embodiment, the nucleic acid molecules can be fragmented and approximately >200 nt. size fragments retained inside the microcapsules. The fragmentation of nucleic acid in a cell lysate within SPM may be performed enzymatically, e.g. using transposase, mixture of nucleases, hydrolases, ultrasound, etc.

In a more specific embodiment, the RNA molecules present in SPM carrying a single-cell lysate are treated with poly(A) Polymerase I to polyadenylate the 3′-termini of the nucleic acid molecules.

In another specific embodiment, the nucleic acid molecules present in SPM carrying a single-cell lysate are treated with ligase to attach oligonucleotides to the 3′-termini of the nucleic acid molecules.

In one embodiment, the SPMs carrying single-cell lysates including nucleic acids are encapsulated in microfluidic droplets along with barcoding DNA oligonucleotides and assay reagents.

In another embodiment, the SPMs carrying single-cell lysates are co-encapsulated along with hydrogel bead carrying barcoding DNA oligonucleotides.

In a more specific embodiment, the SPMs carrying single-cell lysates are co-encapsulated along with hydrogel beads carrying barcoding DNA oligonucleotides and assay reagents.

In some cases, the hydrogel bead comprises barcoding DNA oligonucleotides covalently attached to it.

In a more specific embodiment, the barcoding DNA oligonucleotides may be released from the beads (e.g., by dissolving the beads using reducing agent such as DTT), by using photo-illumination, or by using enzymatic reaction).

In a more specific embodiment, the co-encapsulation of a SPM carrying a cell lysate and a hydrogel bead carrying barcoding DNA oligonucleotides is performed on a microfluidics chip.

In another specific embodiments, at least about 50% of the droplets contain one SPM and one hydrogel bead.

In another specific embodiments, at plurality of droplets contain more than one SPM, and one hydrogel bead.

In another aspect, a SPMs and a bead are co-encapsulated in a droplet along with enzymes and reagents required for attaching the oligonucleotides to nucleic acids molecules.

In a more specific embodiment, the SPMs and beads are co-encapsulated in droplets along with reverse transcription reaction reagents.

In one aspect, a plurality of the nucleic acid molecules within a droplet are being bound to barcoding oligonucleotides during enzymatic reaction. In certain embodiments, the barcoding oligonucleotides within the droplet is distinguishable from barcoding oligonucleotides within the other droplets.

In a more specific embodiment, the SPM loaded in a droplet is dissolved in order to release the encapsulated content of the SPM.

In another aspect, the present invention is generally related to a method. In one set of embodiments, the method includes process of encapsulating a cell in a SPM, lysing the cell within the SPM, the SPM comprising a single-cell lysate including nucleic acids, co-encapsulation of a SPM containing a single-cell lysate with a hydrogel bead and assay reagents within a microfluidic droplet, the hydrogel bead comprising barcoding oligonucleotides covalently attached thereto, and tagging the nucleic acid molecules by the barcoding DNA oligonucleotides within the droplet.

In another aspect, the present invention is generally related to a method where the method includes a process of encapsulating a cell in a SPM, lysing the cell within the SPM, the SPM comprising a single-cell lysate including nucleic acids, fragmenting nucleic acid molecules, and co-encapsulation of SPMs carrying fragmented nucleic acid molecules along with a hydrogel bead and assay reagents within a microfluidic droplet, the hydrogel bead comprising barcoding oligonucleotides covalently attached thereto, and tagging the fragmented nucleic acid molecules by the barcoding oligonucleotide tags within the droplet.

In another aspect, the present invention is generally related to a method where the method includes a process of encapsulating a cell in a SPM, lysing the cell within the SPM, the SPM comprising a single-cell lysate, modifying the nucleic acid molecules, co-encapsulating the SPMs carrying modified nucleic acid molecules along with a hydrogel bead and assay reagents within a microfluidic droplet, the hydrogel bead comprising barcoding oligonucleotides covalently attached thereto, and tagging the modified nucleic acid molecules by the barcoding oligonucleotide tags within the droplet.

The method, in another set of embodiments, includes acts of providing a plurality of SPMs containing cell lysates including nucleic acids, at least about 90% of the SPMs containing a cell lysate originating from a single-cell, or no cell lysate, co-encapsulating SPM along with a hydrogel bead carrying barcoding DNA oligonucleotides and along with assay reagents within a microfluidic droplet and tagging the nucleic acid molecules with DNA barcoding oligonucleotides during enzymatic reaction.

In accordance with one set of embodiments, the method includes acts of co-encapsulating a SPM and a hydrogel bead within a droplet, where the SPMs carry a single-cell lysate and hydrogel bead has attached thereto barcoded oligonucleotides, and enzymatically labelling the RNA and/or DNA with the barcoded oligonucleotides with a microfluidic droplet.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Nor is the claimed subject matter limited to implementations that solve any or all of the disadvantages noted herein.

Other advantages and novel features of the present invention will become apparent from the following detailed description of various non-limiting embodiments of the invention when considered in conjunction with the accompanying figures.

### BRIEF DESCRIPTION OF DRAWINGS

Non-limiting embodiments of the present invention will be described by way of example with reference to the accompanying figures, some of which are schematic and in some cases are not intended to be drawn to scale. For purposes of clarity, not every component is labeled in every figure, nor is every component of each embodiment of the invention shown where illustration is not necessary to allow those of ordinary skill in the art to understand the invention. In the figures:

FIG. 1 provides a schematic showing the state-of-the-art approach for single-cell transcriptomics. Suspension of primary cells are encapsulated into microfluidics droplets with DNA barcoding hydrogel beads and lysis/assay reagents. After encapsulation, DNA primers attached to the hydrogel beads are released and incorporated into cDNA during reverse transcription (RT) reaction. Next, droplets are broken, the barcoded transcriptomes are pooled, amplified and sequenced. The cell barcodes incorporated into cDNA are deconvoluted computationally to obtain digital expression matrix.

FIG. 2 provides a schematic showing an example of the approach of the invention to the performance of single-cell transcriptomics. Cells are encapsulated (#1) in semi-permeable microcapsules (SPMs) such that on average there is one or fewer cells per SPM. The cells are lysed (#2) to generate SPMs containing single-cell lysate. The mRNA within SPMs is further cleaned to remove potential inhibitors. The SPMs carrying single-cell lysates are co-encapsulated (#3) along with hydrogel bead carrying DNA barcoding oligonucleotides and assay reagents. After encapsulation, DNA primers attached to the hydrogel beads are released and incorporated into cDNA during reverse transcription (RT) reaction (#4). Following RT reaction, the droplets are broken (#5), the barcoded transcriptomes are pooled, amplified and sequenced (#6). The cell barcodes incorporated into cDNA are deconvoluted computationally to obtain digital expression matrix (#7).

FIG. 3 provides schematics and operation of microfluidics system for generation of SPMs. 1—an inlet for aqueous phase enriched in shell-forming compound; 2—an inlet for aqueous phase enriched in core-forming compound; 3—carrier oil, 4—emulsion collection outlet. (A) Schematics of microfluidics chip and its operation. (B) Example of the microfluidics chip for the generation of SPMs. (C) Digital micrographs. Scale bars, 100 μm.

FIG. 4 illustrates generation of SPMs and encapsulation of biological species in an example of the invention. A mixture of cells is encapsulated in aqueous two-phase system (ATPS) droplets such that a droplet preferably contains, on average, 1 or 0 cells. The ATPS droplets are converted to semi-permeable microcapsules (SPMs) through a polymerization process. The SPMs dispersed in aqueous buffers remain intact and retain encapsulated cells.

FIG. 5 illustrates schematics and operation of microfluidics system for co-encapsulating SPMs and hydrogel beads into microfluidic droplets in an example of the invention. (A) Schematics of microfluidics chip and its operation. 1—hydrogel beads carrying barcoding DNA oligonucleotides; 2—semi-permeable microcapsules (SPMs) carrying nucleic acids originating from single-cells; 3—a microchannel for delivering assay reagents, 4—a microchannel for delivering the carrier oil, 5—SPM and hydrogel bead co-encapsulation in microfluidic droplet. Once droplets are collected the resulting emulsion will contain plurality of droplets where majority of droplets may contain one SPM and one hydrogel bead (#6), and some droplet will contain one hydrogel bead (#7), one SPM (#8) or none (#9). Other combinations such as two hydrogel beads in one droplet, or two SPMs in one droplet are also possible but are undesirable. (B) Still micrograph during the operation of a system, in accordance with one example of an embodiment of the invention. The SPMs carrying single-cell lysates co-encapsulation in microfluidic droplets along with hydrogel beads carrying barcoding oligonucleotides, and along with assay reagents needed for labelling the nucleic acids with said DNA barcodes. Scale bars, 100 μm.

FIG. 6 presents design of microfluidics system for co-encapsulating SPMs and hydrogel beads into microfluidic droplets in an example of the invention. 1—an inlet for hydrogel beads; 2—an inlet for SPMs; 3—an inlet for assay reagents, 4—an inlet for carrier oil, 5—emulsion collection outlet.

FIG. 7 illustrates droplets carrying SPMs and hydrogel beads collected off-chip. Droplets carrying co-encapsulated SPMs, hydrogel beads and assay reagents were collected off-chip and imaged under bright field microscope. Scale bar, 100 μm.

FIG. 8 illustrates the profile of amplified barcoded cDNA of mammalian cells. The SPMs carrying lysed mammalian cells and hydrogel beads carrying barcoding oligonucleotides were co-encapsulated in microfluidic droplets as shown in FIG. 5. The barcoded cDNA generation and amplification are detailed in the main text. Trace indicated with solid arrows represents barcoded cDNA profile, where cDNA barcoding reaction was performed on encapsulated cells (meaning that no SPMs were used); Trace indicated with curly arrows represents barcoded cDNA profile where the SPMs carrying cell lysates were used and where the RT reaction mix was supplemented with collagenase enzyme (0.4 mg/ml); Trace indicated with dashed arrows represents barcoded cDNA profile where the SPMs carrying cell lysates were used and where the collagenase enzyme was excluded from RT reaction mix.

FIG. 9 illustrates the final DNA library profile constructed from barcoded mammalian cDNA species. Trace indicated with solid arrows represents DNA library profile, where cDNA barcoding reaction was performed on encapsulated cells (no SPMs used); Trace indicated with curly arrows represents DNA library profile where the SPMs carrying cell lysates were used and where the RT reaction mix was supplemented with collagenase enzyme (0.4 mg/ml); Trace indicated with dashed arrows represents DNA library profile where the SPMs carrying cell lysates were used and where the collagenase enzyme was excluded from RT reaction mix.

FIG. 10 illustrates an example experimental approach for single-cell transcriptomics including nucleic acid modification step. Cells, for example bacteria cells, are encapsulated (#1) in semi-permeable microcapsules (SPMs) such that on average there is one, or fewer cells, per SPM. The cells are lysed (#2). The SPMs containing single-cell lysate are treated to remove undesirable inhibitors, proteins, lysis reagents, etc. The SPMs carrying nucleic acid molecules such as RNA molecules are further treated to modify the encapsulated nucleic acids. For example, RNA molecules can be modified by polyadenylation, RNA molecules can be modified by ligating DNA or RNA adapters, and modified in other ways. The SPMs carrying modified nucleic acid molecules are co-encapsulated (#4) along with hydrogel bead carrying DNA barcoding oligonucleotides and assay reagents. After encapsulation, DNA primers attached to the hydrogel beads are released and incorporated into modified RNA, for example, during reverse transcription (RT) reaction (#5). Following RT reaction, the droplets are broken (#6), the barcoded transcriptomes are pooled, amplified and sequenced (#7). The cell barcodes incorporated into cDNA are deconvoluted computationally to obtain digital expression matrix (#8).

FIG. 11 illustrates digital photographs of microfluidic droplet generation for nucleic acid barcoding. Co-encapsulation of SPMs carrying nucleic acids derived from single-cells, hydrogel bead carrying barcoding DNA oligonucleotides and assay reagents in microfluidic droplets using a microfluidics chip indicated in FIG. 6. Adjusting the flow rates of the system it is possible to obtain plurality of droplets having one SPM and one hydrogel bead as well as having several SPMs and one hydrogel bead. A—majority of droplets contain one SPM and one hydrogel bead. Left photograph shows SPM and hydrogel bead co-encapsulation step, right image shows droplet collection. B—majority of droplets contain multiple SPMs and one hydrogel bead. Left photograph shows SPM and hydrogel bead co-encapsulation step, right image shows droplet collection. Scale bars, 20 μm.

FIG. 12 illustrates the barcoded bacterial cDNA profile after PCR; The amplified cDNA was released from droplets and purified twice with 0.6× volume AMPure magnetic beads.

FIG. 13 illustrates the final DNA library profile constructed from barcoded bacterial cDNA species. The amplified DNA library purified using double size selection (0.6-0.8× SPRI beads).

FIG. 14 shows an example experimental approach for single-cell genomics of bacterial cells. Bacteria cells are encapsulated (#1) in semi-permeable microcapsules (SPMs) such that on average there is one or fewer cells per SPM. The cells are lysed (#2) to generate SPMs containing single-cell lysate. The DNA within SPMs is fragmented. The SPMs carrying fragmented DNA are co-encapsulated along with hydrogel beads carrying DNA barcoding oligonucleotides and assay reagents (#3). After encapsulation, DNA primers attached to the hydrogel beads are released and are attached to fragmented DNA (#4). The barcoded DNA fragments are released from droplets (#5). The barcoded DNA fragments are pooled, amplified and sequenced (#6). The cell barcodes attached to fragmented DNA are deconvoluted computationally to obtain digital expression matrix (#7).

FIG. 15 shows an example experimental approach for single-cell genomics of mammalian cells. Mammalian cells are encapsulated in semi-permeable microcapsules (SPMs) such that on average there is one or fewer cells per SPM (#1). The cells are lysed to generate SPMs containing single-cell lysates and the DNA within SPMs is fragmented (#2). The SPMs carrying fragmented DNA are co-encapsulated along with hydrogel beads carrying DNA barcoding oligonucleotides and assay reagents (#3). After encapsulation, DNA primers attached to the hydrogel beads are released and are attached to fragmented DNA (#4). The barcoded DNA fragments are released from droplets (#5). The barcoded DNA fragments are pooled, amplified and sequenced (#6). The cell barcodes attached to fragmented DNA are deconvoluted computationally to obtain digital expression matrix (#7).

FIG. 16 shows an example experimental approach for single-cell epigenomics and transcriptomics. Cells are encapsulated in semi-permeable microcapsules (SPMs) such that on average there is one or fewer cells per SPM. The cells are lysed to generate SPMs containing single-cell lysates with mRNA and chromatin (#1). The chromatin DNA is fragmented within SPM (#2). The SPMs carrying fragmented genomic DNA (gDNA) and mRNA molecules are co-encapsulated along with hydrogel beads carrying DNA barcoding oligonucleotides and assay reagents (#3). After encapsulation, DNA primers are released from the hydrogel beads and bind to fragmented DNA and to mRNA followed by a nucleic acid barcoding reaction (#4). The barcoded gDNA and copy DNA (cDNA) fragments are released from droplets (#5). The barcoded nucleic acid fragments are pooled, amplified and sequenced (#6). The cell barcode sequences of nucleic acids are deconvoluted computationally to obtain digital expression matrix (#7).

FIG. 17 shows a schematic of an example experimental approach for single-cell methylomics. Cells are encapsulated in semi-permeable microcapsules (SPMs) such that on average there is one or fewer cells per SPM. The cells are lysed to generate SPMs containing single-cell lysates, and genomic DNA is fragmented within the SPM (#1). The SPMs carrying fragmented genomic DNA (gDNA) are co-encapsulated along with hydrogel beads carrying DNA barcoding oligonucleotides and assay reagents (#2). After encapsulation, barcoding DNA primers are released from the hydrogel beads and are attached to fragmented DNA by a nucleic acid barcoding reaction (#3). The barcoded gDNA fragments are released from droplets (#4). The DNA modifications are converted to another base (#5), such as for example methyl-cytosines (methyl-Cyt) are converted to another to another base, dihydrouracil (DHU). The barcoded nucleic acid fragments are amplified and sequenced (#6). The cell barcodes are deconvoluted computationally to obtain digital expression matrix (#7).

FIG. 18 provides a UMAP representation of single-cell transcriptomes prepared using different methodologies. Single-cell RNA-Seq libraries were prepared as described in the Example 9 and showed no significant technical biases of the nucleic acid barcoding method disclosed in this invention.

FIG. 19 provides a UMAP representation of human PBMC prepared using different methodologies. Single-cell RNA-Seq libraries were prepared as described in the Example 9. The results confirmed that the method of this disclosure enables accurate identification of cell types in a biological sample based on their gene expression signatures.

FIG. 20 illustrates the barcoded bacterial cDNA profile after PCR; The amplified cDNA was released from droplets and purified twice with 0.6× volume AMPure magnetic beads. The solid and dashed lines indicate the barcoded cDNA obtained at different experiments.

FIG. 21 illustrates the final DNA library profile constructed from barcoded bacterial cDNA species. The amplified DNA library purified using double size selection (0.6-0.8× SPRI beads). The solid and dashed lines indicate the final DNA library obtained at different experiments.

FIG. 22 illustrates single-cell RNA-Seq results as a species-mixing plot. Where on Y-axis is total transcript count from B. subtilis cells and X-axis is the total transcript count from E. coli cells. Sequencing reads that align to both genomes (e.g. when two cells of different species were barcoded with the same cell barcode) is indicate as “mixed” cluster.

### DETAILED DESCRIPTION OF THE INVENTION

Throughout the text the terms of ‘comprising’ and ‘containing’ have been used interchangeably and have the same meaning.

The terms “approximately” or “about” are used herein and generally refer to a range of +30% of the stated value, preferably +20% of the stated value, more preferably +10% of the stated value, and even more preferably +5% of the stated value.

Articles such as “a,” “an,” and “the” include the singular and the plural reference unless the context clearly indicates otherwise. For example, a reference to “a cell” includes one cell and two or more of such cells.

The present invention generally relates to methods of nucleic acid processing and analysis, and in particular in one aspect to a method of labelling nucleic acids of a cell lysate derived from a single-cell. Certain aspects are generally directed to methods for generating a cell lysate within a semi-permeable microcapsule (SPM).

As noted above the present invention is directed to a method comprising a step of co-encapsulating a microcapsule and a particle in a droplet.

In particular, the present invention involves microcapsules comprising a core surrounded by a semi-permeable shell. As noted above, such microcapsules are also referred to herein as “semi-permeable microcapsules” or “SPM”.) The SPMs are approximately 10 to 1000 μm sized compartments.

The methods of the present invention may comprise a step of encapsulating the biological species in the microcapsule, and in particular, may comprise a step of encapsulating a plurality of biological species into a plurality of microcapsules. In particular, a mixture of cells may first be encapsulated in a plurality of microfluidic droplets. Those of ordinary skill in the art will be aware of techniques for encapsulating cells within microfluidic droplets [27, 28]; see also for example, U.S. Pat. Nos. 7,708,949, 8,337,778, 8,765,485 each incorporated herein by reference. The cells preferably are encapsulated at a density such that on average one droplet would contain 1 cell or less. Also encapsulated in the droplets is polymer solution that forms aqueous two-phase system (ATPS). In non-limiting example, the ATPS droplets are converted to SPMs by inducing the sol-gel transition and solidification of the shell followed by a cross-linking reaction (e.g. photo-polymerization).

Methods for encapsulating cells in microcapsules having a semi-permeable shell and a core are described in US 2020/0400538 A1 (which describes encapsulation of cells in microcapsules made in particular from PEGDA and dextran), and provisional application U.S. 63/284,657. In particular, as described further below, and as demonstrated in Example 1, suitable microcapsules may be made with a polyampholyte (gelatin methacrylate) and a polysaccharide (dextran).

In one set of embodiments, the cells are loaded such that, on the average, each SPM has less than 1 cell in it. For example, the average loading rate may be less than about 1 cell/SPM, less than about 0.9 cells/SPM, less than about 0.8 cells/SPM, less than about 0.7 cells/SPM, less than about 0.6 cells/SPM, less than about 0.5 cells/SPM, less than about 0.4 cells/SPM, less than about 0.3 cells/SPM, less than about 0.2 cells/SPM, less than about 0.2 cells/SPM, less than about 0.1 cells/SPM, less than about 0.05 cells/SPM, less than about 0.01 cells/SPM.

The present invention makes use of such microcapsules to carry or retain nucleic acids obtained from a biological species, so that the nucleic acids can be co-encapsulated in a droplet with a particle comprising (or carrying) one or more reagents for use in the processing of the nucleic acid as specified above.

Once formed around a biological species such as a cell, the microcapsule is able to retain the biological species and nucleic acid molecules obtained/released from the biological species, e.g. by disrupting (lysing) a cell or by degrading or digesting proteins closely associated with the nucleic acid inside the microcapsule. In particular, double stranded nucleic acids longer than approximately 200 nucleotides, preferably longer than approximately 150 nucleotides, and more preferably longer than approximately 100 nt., can be retained in the microcapsule, but the shell of the microcapsule is permeable to low molecular weight compounds such as salts, proteins, enzymes and DNA oligonucleotides, and allows such compounds to diffuse into and out of the core. Accordingly, a microcapsule comprising nucleic acid obtained from a biological species can be produced, and as discussed further below, the nucleic acid can be subjected to further processing steps, e.g. amplification, reverse transcription of RNA to cDNA, fragmentation etc., before the microcapsule comprising the nucleic acid is co-encapsulated with the particle in the droplet.

A biological species as referred to herein is the source of the nucleic acid and can be a cell, a microorganism, a bacterium, or a virus. In addition, the biological species may be a cell-free biological sample, such as a blood sample taken for cell-free DNA (cfDNA) testing, e.g. prenatal cell-free DNA testing or circulating tumor DNA testing. While these nucleic acids samples are cell-free they may be associated with e.g. proteins and their release therefrom may be desirable to improve the efficacy of e.g. barcoding steps. However, it is preferred that the biological species is a cell, either a prokaryotic or a eukaryotic cell. Preferably the cell is a eukaryotic cell, more preferably a mammalian cell and most preferably a human cell.

Nevertheless, although the biological species being a cell is a preferred embodiment (and cells are the main source of the nucleic acid material), and in addition for ease of reference the descriptions below refer to the practice of the methods of the invention with the biological species being a cell, it will be appreciated that the methods of the invention can equally be performed with other biological species, e.g. the nucleic acid may be from other sources such as bacteria, viruses or microorganisms.

In some embodiments, the microcapsules (or the majority of microcapsules if a plurality of microcapsules are being used) may comprise a single biological species, e.g. a single cell, or nucleic acid obtained therefrom. However, in other embodiments, the microcapsules (or the majority of microcapsules if a plurality thereof are being used) may comprise two or more biological species, or nucleic acid obtained therefrom. The two or more biological species can be the same biological species, i.e. two or more cells of the same or different origin, type or sub-type, or different biological species, e.g. a cell and a virus.

In some embodiments the methods of the invention use microcapsules comprising nucleic acid obtained from one or more biological species, and which nucleic acid has already been released into the core of the microcapsule. In other embodiments, the methods may comprise the steps of encapsulating the one or more biological species into the microcapsule and optionally a step of releasing the nucleic acid, or other material from the biological species, into the core of the microcapsule, preferably wherein the nucleic acid is released inside the microcapsule by a step of disrupting (lysing) the cell inside the microcapsule prior to the co-encapsulation step described herein. This method step may be performed by dispersing the microcapsules carrying cells in an aqueous solution containing lysis reagents. For example, the cells may be lysed using chemicals (e.g. SDS), enzymatic (e.g. lysozyme) or physical (e.g. ultrasound) means. The cells may release nucleic acids such as DNA and/or RNA as well as proteins, enzymes and other biomolecules. This step may be followed by dispersing the microcapsules in an aqueous solution to replace the lysis reagents, e.g. with other reaction components or salts suitable for the next process step. After lysis, part of the cellular material (e.g. proteins, lipids, metabolites) may passively diffuse out from the interior part of the microcapsule when suspended in an aqueous buffer. In a preferred embodiment of the methods described herein microcapsules comprising cell lysates may be treated in order to purify the released nucleic acids and/or deplete one or more enzymatic inhibitors in the lysate, which may be detrimental to the efficiency of the process step that is to be performed in the droplet, e.g. attaching molecular tags, and in particular barcodes, to the nucleic acids.

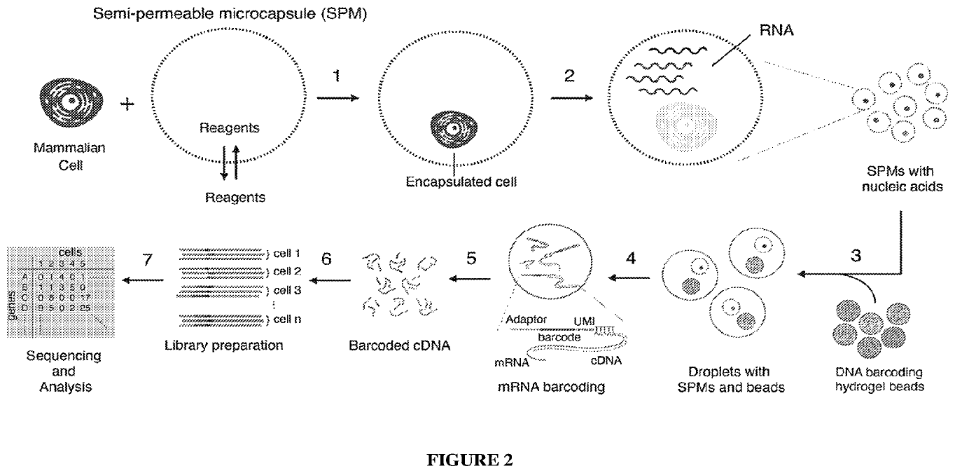

In some cases, the nucleic acids that are released may be subjected to further processing performed inside the microcapsule, for example, by including suitable reagents specific to the nucleic acid processing method. Examples of nucleic processing known to those of ordinary skill in the art include, but are not limited to, fragmentation, poly(A) tailing, ligation to an oligonucleotide, reverse transcriptase (RT) primer extension reaction, polymerase chain reaction (PCR), multiple displacement amplification (MDA), or 5′- or 3′-end, or both, modification. In further examples, the microcapsule containing e.g. a single-cell lysate, may be dispersed in an aqueous buffer to initiate, modify or terminate a desirable enzymatic or chemical reaction. Such steps may be performed before co-encapsulation of the microcapsule with the particle into the droplet so as to take advantage of the ability for buffer/reagent exchange through the shell of the microcapsule. Alternatively, as in one non-limiting example of the invention that is shown in FIG. 2, the cell is lysed within a microcapsule to release RNA, and further processing steps before labelling such as reverse transcription can be performed in the droplet after co-encapsulation with the particle.

The particle that is co-encapsulated into the droplet with the microcapsule may comprise a reagent for use in the processing of the nucleic acid, for example a molecular tag or label, such as a nucleic acid tag or a barcode, an enzyme, an antibody, a lytic reagent, a DNA primer, or a dye. In a preferred embodiment of the methods of the invention the particle comprises an oligonucleotide comprising a barcode.

In particular, a barcode is a user-defined DNA sequence preferably longer than 4 nucleotides but shorter than 100 nucleotides and more preferably in the range of 6-70 nucleotides and even more preferably in the range of 8-16 nucleotides long.

In the methods of the invention discussed herein, where nucleic acid from a plurality of cells is being barcoded, the diversity of unique barcode sequences is at least 100, and more preferably more than 1000, and more preferably more than 10,000 and more preferably more than 100,000 and even more preferably more than 1,000,000 but less than 10\hat{ }12.

In a preferred embodiment the barcode is comprised in a barcoding DNA oligonucleotide which is 12 to 300 nucleotides in length, preferably 20 to 150 nucleotides in length, and more preferably 30 to 120 nucleotides in length. The barcoding DNA oligonucleotide may further comprise one or more of (i) a unique molecular identifier (UMI), wherein the UMI is a random nucleotide sequence longer than 4 nucleotides but shorter than 50 nucleotides, and preferably in the range of 4-12 nucleotides and still more preferably 8 to 16 nucleotides in length; (ii) a cell barcode preferably longer than 4 but shorter than 100 nucleotides, and more preferably 6 to 70 nucleotides in length, (iii) a sequence able to specifically bind to a region of interest in the nucleic acid (e.g. a poly-T sequence or a gene specific sequence); (iv) an overhang, (e.g. a sticky end) or a blunt end; and (v) an adapter sequence (e.g. a PCR adapter and/or a sequencing adapter and/or hybridization adapter).

The particles used for co-encapsulation may be solid particles comprising a molecular tag or squishy particles (hydrogel beads) comprising a molecular tag [13]. When a plurality of droplets are formed co-encapsulating a plurality of microcapsules and a plurality of solid particles, typically 1 to 33% of droplets will end up comprising one microcapsule and one solid particle. When a plurality of droplets are formed co-encapsulating a plurality of microcapsules and a plurality of squishy particles (e.g., hydrogel beads), typically >30% and even >50% of the droplets will end up comprising one microcapsule and one hydrogel bead.

The particle may be hard or soft, it can be made of organic or inorganic material, it can be a solid particle, a hydrogel particle, a hydrogel bead, or composite hydrogel bead. It may also comprise polyacrylamide, agarose, polystyrene and/or poly-N-isopropylacrylamide. Preferably the particle has a size of in the range of 1-100 μm and preferably in the range of 10-80 μm, and more preferably in the range of 20-70 μm, and more preferably approximately 60 μm.

The particle is preferably a hydrogel particle. However, while many of the embodiments discussed below refer to the use of hydrogel particles, it will be appreciated that other particles may also be used.

It is preferred that the reagent, and in particular the molecular tag or barcoding DNA oligonucleotide referred to herein, is covalently attached to the particle. However, this is not essential and in other examples, the particle may comprise a short single stranded nucleotide stub to which the reagent base-pairs.

Where the attachment is covalent, it may comprise a cleavable linker, such as a photocleavable linkers, a chemically cleavable linker, or an enzymatically cleavable linker, such that the reagent and in particular the molecular tag or barcoding DNA oligonucleotide referred to herein can be released from the particle by exposing the plurality of droplets to light (preferably below 450 nm wavelength), to a chemical agent (e.g. a reducing agent), or an enzyme (e.g. endonuclease), respectively, to release the molecular tag covalently attached to the particle when the particle is in the droplet with the microcapsule.

However, it should be noted that the methods described herein do not always involve a step of releasing the reagent (e.g. the molecular tag or barcoding DNA oligonucleotide) from the particle. In particular, the reagent may remain attached to the particle inside the droplet, while the microcapsule is broken inside the droplet to release the nucleic acid from the core and allow it to come into contact with the reagent. In other embodiments the particle may comprise more than one reagent. In an example of such an embodiment, the particle may comprise two molecular tags, e.g. one barcoding DNA oligonucleotide that is to be attached to DNA released from the cell in the microcapsule, and one barcoding DNA oligonucleotide that is to be attached to cDNA produced with a reverse transcriptase reaction with the RNA released from the cell in the microcapsule. In such an embodiment it may be desirable for the barcoded DNA to become attached by the barcoding DNA oligonucleotide to the particle, while the barcoded RNA is untethered, or vice versa.

The microcapsule comprising the nucleic acid and the particle comprising the reagent/molecular tag, are co-encapsulated in a droplet. The droplet may be a microfluidic droplet, i.e. a droplet generated with a microfluidic device. Preferably the droplet is an oil-in-water droplet generated using a microfluidic device.

Such microfluidic droplets have particular utility in high throughput methods involving a large number of samples of biological species and in particular a large number of cells. Accordingly, the method of the present invention can be readily scaled to attach barcodes to a large number of samples.

In one set of embodiments, the SPMs can be loaded into water-in-oil droplets together with hydrogel beads and assay reagents. The SPMs suspended in aqueous buffer can be packed (concentrated) such that their delivery into droplets becomes ordered and synchronized, enabling loading of a desirable number of SPMs into a droplet, such as exactly one SPM, two SPMs, etc. Likewise, the hydrogel beads carrying barcoding DNA primers suspended in aqueous buffer can also be packed (concentrated) such that their delivery into droplets becomes ordered and synchronized, ensuring that the majority of droplets host exactly one hydrogel bead. As a result, the co-delivery of a SPM and a hydrogel bead into the same droplet can be precisely controlled and high co-occupancy events (one SPM and one hydrogel bead) can be achieved. In a typical scenario over 50% of droplets will contain one SPM and one hydrogel bead.

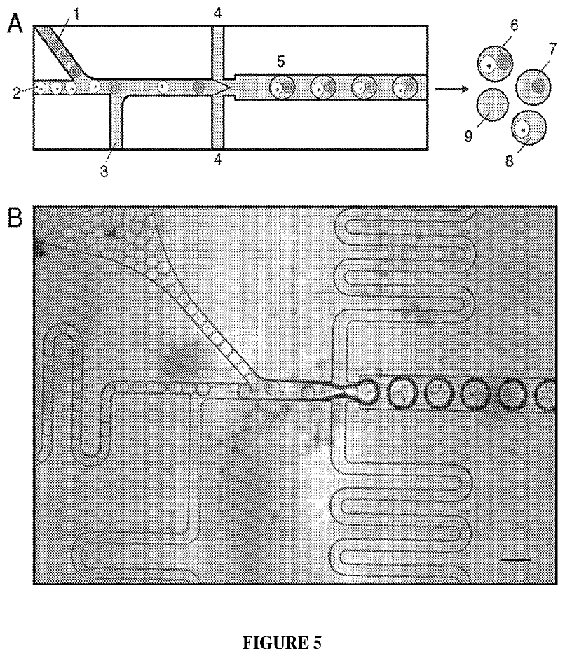

One non-limiting example of the invention is shown in FIG. 5. A suspension containing a plurality SPMs is injected into a microfluidics chip along with plurality of hydrogel beads and assay reagents. Those of ordinary skill in the art will be aware of techniques for loading hydrogel beads in microfluidic droplets and for co-encapsulating cells within microfluidic droplets with hydrogel beads [13, 15, 30]; see also for example, U.S. Pat. 10596541 incorporated herein by reference. The SPMs carrying cells, and more preferably carrying single-cell lysates are introduced to a microfluidics chip at such flow rates that each microfluidic droplet would preferably contain one SPM. Simultaneously, the hydrogel beads are injected into a microfluidics chip at such flow rates that each microfluidic droplet would preferably contain one bead. Therefore, adjusting the flow rates it is possible to achieve conditions where most of the droplets will contain one SPM and one bead.

In some embodiments, the encapsulation conditions are chosen such that droplets contain one SPM and one hydrogel bead. The presence of empty droplets and/or droplets with single hydrogel beads but without SPMs, and/or droplets with SPMss but without hydrogel beads, do not substantially affect performance. The co-encapsulated SPMs and hydrogel beads may be collected in the form of an emulsion and processed according to the aim of the particular application. For example, in one particular embodiment, the RNA of single-cell lysates is converted into barcoded complimentary DNA upon reverse transcription or other DNA polymerization reaction with barcoded oligonucleotides i.e., introduced with a hydrogel bead. In another particular embodiment, the DNA of single-cell lysates is converted into barcoded DNA upon DNA polymerization reaction with barcoded oligonucleotides i.e., introduced with a hydrogel bead. In yet another particular embodiment, the DNA of single-cell lysates is converted into barcoded DNA upon ligation reaction with barcoded oligonucleotides i.e., introduced with a hydrogel bead.

In one set of embodiments, the nucleic acid molecules may be “barcoded” or include unique sequences attached that can be used to distinguish nucleic acids in a droplet from those in another droplet, or even when the nucleic acids are pooled together. The barcoded oligonucleotides having unique sequences are delivered to individual droplets by hydrogel beads attached thereto and the nucleic acids containing within the droplets (for example, those present in a cell lysate) are “barcoded” during enzymatic reaction by barcoded oligonucleotides. The barcodes are used to distinguish nucleic acids, e.g., originating from different cell lysates. The barcoded oligonucleotides attached to nucleic acid molecules within a droplet may be distinguishable from oligonucleotide tags in other droplets. Barcoding nucleic acid molecules is important when pooling the nucleic acids from different droplets.

In another set of embodiments, the barcoding DNA oligonucleotide are introduced into the droplets by a hydrogel bead or a solid particle and then released from the bead once the bead is loaded into a droplet. When the hydrogel beads are loaded within the droplets at a density such that one droplet on average contains no more than 1 bead, then once the barcoding DNA oligonucleotide are released from the bead, then most or all of the droplets will contain one unique barcoding DNA oligonucleotide, thus allowing each droplet (and the nucleic acids contained therein) to be uniquely labeled and identified.

It should be understood that the barcoding DNA oligonucleotide introduced into the droplets by a bead (hydrogel bead or a solid particle) may remain attached to the bead once the bead is loaded into a droplet. When the barcoding DNA oligonucleotide remains attached the bead, the nucleic acids (released by lysed cells) will be captured by the said barcoding DNA oligonucleotide attached to the bead.

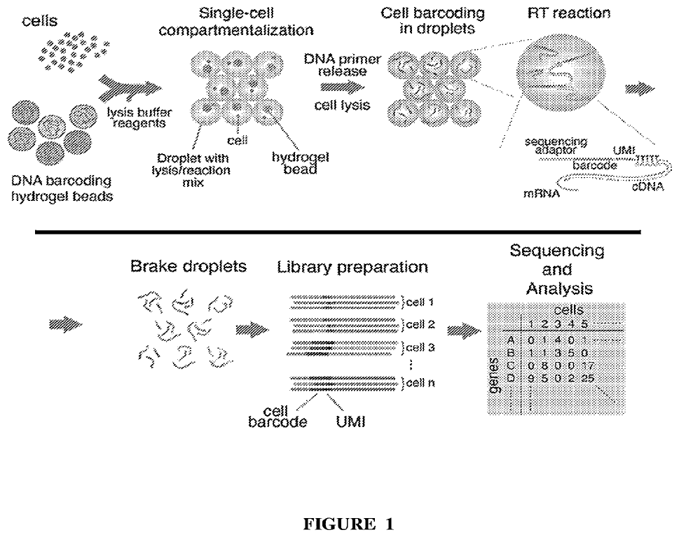

It should be understood that the barcoding oligonucleotides are initially attached to hydrogel beads in order to facilitate the introduction of only one unique barcoded oligonucleotide type to each droplet, as is shown in FIGS. 1, 2, 5, 7 and 11. After introducing a hydrogel bead with barcoding oligonucleotides to droplet and releasing them, a droplet will contain a plurality (typically in other range of 10\hat{ }3-10\hat{ }9) of barcoded oligonucleotides containing the same unique barcode. Therefore, each barcoding oligonucleotide present within a droplet will be distinguishable from the barcoding oligonucleotide present in the other droplets.

To release barcoding oligonucleotides from the hydrogel-bead a light at approximately 400 nm wavelength range may be applied to cleave the photolabile bond and release barcoding oligonucleotides from the hydrogel. However, it should be understood that this is an example only, and that other methods of cleavage or release can also be used, for example by melting the hydrogel bead with reducing agent [15]. In one set of embodiments, agarose particles containing oligonucleotides may be used, and the oligonucleotides may be released by heating the agarose, e.g., until the agarose at least partially liquefies or softens. However, in some other embodiments, cleavage may be nonessential.

In another set of embodiments, the droplets may be broken to release the barcoded nucleic acids and other contents. The barcoded nucleic acids may then be pooled together and since the barcoded nucleic acids molecules are labeled with different barcoded oligonucleotide tags, the nucleic acids from one droplet (i.e., from one SPM) can be distinguished from those from other droplets (i.e. from other SPMs) by the sequencing of barcoded oligonucleotide tags.

In another aspect, the present invention provides systems and methods for the massively parallel barcoding of DNA or RNA from large numbers of single-cell lysates. This process may rely on the co-encapsulation of SPMs carrying single-cell lysates along with barcoded nucleic acids, or other suitable oligonucleotide tags attached to hydrogel or polymer beads, together with other reagents that may be used for RNA and/or DNA capture, and/or extension and/or amplification.

In one set of embodiments, the nucleic acid contents of each cell lysate present in droplet may be labeled with a unique barcode and may allow for hundreds, thousands, or millions of cell lysates to be barcoded in a single experiment for the purpose of determining the cell composition in a population or for screening cell populations.

In yet another aspect, the present invention provides systems and methods for the massively parallel capture, barcoding and quantification of nucleic acid molecules from a large number of single cells, for the purpose of profiling cell populations, characterizing their transcriptome, characterizing their genome, or other purposes.

After purification and optional DNA amplification, the base composition and barcode identity of cellular nucleic acids may be determined, for instance, by sequencing. Alternatively, in some embodiments, DNA oligonucleotides introduced with hydrogel beads may serve as primers for amplification of region of interest in the genomic DNA.

In one non-limiting embodiment, the 3′ end of a barcoded oligonucleotide is terminated with a poly-T sequence that may be used to capture cellular mRNA. The 3′ end of poly-T oligonucleotide may serve as a reverse transcription primer and may be extended to create cDNA. In one set of embodiments, the 3′ end of the barcoded primers may terminate with a random DNA sequence that can be used to capture the RNA or DNA of a cell lysate. In another embodiment, the 3′ end of the barcoded primers may terminate with a specific DNA sequence, e.g., that can be used to capture DNA or RNA species (“genes”) of interest, or to hybridize to a DNA probe that is delivered into the droplets in addition to the hydrogel beads, together with the enzyme reagents. In another embodiment, a hydrogel bead may carry a number of different primers to target several genes of interest. Analytical techniques can be used to analyze, for example, genomes, transcriptomes, epigenomes, single nucleotide polymorphisms, specific gene expression levels, non-coding RNA, etc. However, the invention should not be limited to only these applications.

In some cases, systems and methods revealed here are related to barcoding the specific set of genes (e.g., tens, or hundreds or even thousands of genes) of individual cells with a unique barcode and prepare genetic material of hundreds, thousands, or even hundreds of thousands or more of individual cells in a single experiment. For example, in some applications it may be desirable to analyze a sub-set of genes of interest, for example between tens to hundreds of genes, rather than whole-transcriptome or whole-genome sequencing.

In some cases, systems and methods revealed here are related to barcoding the DNA fragments arising from individual cells with a unique barcode, and prepare barcoded genetic material from hundreds, thousands, or even hundreds of thousands or more of individual cells in a single experiment.

Some embodiments of the invention may be used to quantify protein abundance in single cells in parallel to RNA or DNA, for example, by first treating cells with DNA-tagged antibodies as discussed in references [24, 25]. After sequencing, the data may be split according to the barcodes and provide information about the molecule count, origin of nucleic acids and/or proteins of interest.

The above discussions are non-limiting examples of various embodiments of the present invention. However, other embodiments are also possible. Accordingly, more generally, various aspects of the invention are directed to various systems and methods for barcoding nucleic acids within microfluidic droplet carrying a single-cell lysate within SPM and a hydrogel bead carrying barcoded oligonucleotides, as discussed below.

In one aspect, the present invention is generally directed to systems and methods for barcoding nucleic acids within a plurality of microfluidic droplets having an average diameter of the droplet of less than 1000 μm, and more preferably having an average diameter of approximately 100 μm.

The barcoding oligonucleotides may be of any suitable length or comprise any suitable number of nucleotides. The oligonucleotide tags may comprise DNA, RNA, and/or other nucleic acids such as PNA, and/or combinations of these and/or other nucleic acids. In some cases, the oligonucleotide tag is single stranded, although it may be double stranded in other cases. For example, the oligonucleotide tag may have a length of at least about 10 nt, at least about 30 nt, at least about 50 nt, at least about 100 nt, at least about 300 nt, at least about 500 nt, at least about 1000 nt, etc. The length of the barcoding oligonucleotide may vary depending on the application and could be either single-stranded or double-stranded.

As described above, the barcoding oligonucleotides may contain a variety of sequences. For example, the oligonucleotides may contain one or more primer sequences, one or more unique or “barcode” sequences, random sequences, degenerative sequences, one or more promoter sequences, one or more spacer sequences, or the like. Other examples include barcoding oligonucleotides may include a poly-A tail, enzyme recognition sequences, or the like. The oligonucleotide tag may also contain, in some embodiments one or more cleavable spacers, e.g., photocleavable linker. The oligonucleotide tag may be attached to a particle chemically (e.g., via a linker) or physically (e.g., without necessarily requiring a linker).

In addition, in one set of embodiments, a plurality of hydrogel beads, for instance, containing barcoding oligonucleotide tags on their surface may be loaded to droplets, e.g., such that, on average, each droplet contains one hydrogel bead, or less in some cases. After being added to the droplet, the barcoding oligonucleotides may be released from the bead, e.g., using light, reducing agents, or other suitable techniques, to allow the oligonucleotides to become present in solution, i.e., within the interior of the droplet. In such fashion, by loading of the hydrogel beads into the droplets in some embodiments, then releasing the oligonucleotides within the droplet, e.g., to interact with nucleotides or other species, such as is discussed herein.

In one set of embodiments, plurality of droplets is formed in a way that majority of individual droplets would contain a single SPM (with or without single-cell lysate within), and a hydrogel bead comprising barcoding oligonucleotides as described above. A wide variety of different techniques for forming droplets are known to those of ordinary skill in the art. For example, a junction of channels may be used to create the droplets. The junction may be, for instance, a T-junction, a Y-junction, a channel-within-a-channel junction (e.g., in a coaxial arrangement, or comprising an inner channel and an outer channel surrounding at least a portion of the inner channel), a cross (or “X”) junction, a flow-focusing junction, or any other suitable junction for creating droplets. See, for example, [31], WO 2004/091763, WO 2004/002627, each of which is incorporated herein by reference.

In some embodiments, the droplets are loaded with SPMs and hydrogel beads such that, on the average, a large fraction (e.g., >50%) of droplet has 1 SPM and 1 hydrogel bead cell. In some cases, higher or lower loading rates may be chosen to minimize the probability that a droplet will be produced having two or more SPMs in it. Thus, for example, at least about 10%, at least about 20%, at least about 30%, at least about 40%, at least about 50%, at least about 60%, at least about 70%, at least about 80%, at least about 90%, at least about 95%, of the droplets may contain 1 SPM and 1 hydrogel bead.

A relatively large number of droplets may be created, e.g., at least about 100, at least about 500, at least about 1,000, at least about 3,000, at least about 5,000, at least about 10,000, at least about 30,000, at least about 50,000, at least about 100,000 droplets, at least about 300,000 droplets, at least about 500,000 droplets, at least about 1,000,000 droplets, etc.

As discussed, in certain aspects, the droplets may contain nucleic acids delivered to droplets by encapsulated SPMs. The nucleic acid may arise from a cell, or from other biological sources. In one set of embodiments, if SPM is present in a droplet, it may contain a lysed cell e.g., nucleic acid molecules from the cell, etc. In some embodiments, some of the nucleic acids may also be joined to one or more barcoded oligonucleotides contained within the SPM and therefore by extension within the droplet. For example, in one set of embodiments, RNA transcripts typically produced within the cells may be present in SPM and then either released within the droplet or retained within SPM while being converted to cDNA and tagged to the barcoding oligonucleotides.

In yet another set of embodiments, the nucleic acids may be tagged with the barcoding oligonucleotide and/or amplified using PCR (polymerase chain reaction) or other suitable amplification techniques. In one set of embodiments, nucleic acid amplification may be performed within the droplets, within encapsulated SPM or within both SPM and droplet. Those of ordinary skill in the art will be aware of suitable PCR techniques and variations, such as assembly PCR or polymerase cycling assembly, which may be used in some embodiments to produce an amplified nucleic acid. In addition, in some cases, suitable primers may be used to initiate polymerization, or other primers known to those of ordinary skill in the art.

In some cases, the droplets may be broken, or otherwise fused. A wide variety of methods for bursting droplets are available to those of ordinary skill in the art, and the exact method chosen is not critical. For example, droplets contained in a carrying fluid may be disrupted using techniques such as mechanical disruption or ultrasound. Droplets may also be disrupted using chemical agents or surfactants, for example, 1H,1H,2H,2H-perfluorooctanol.

Once droplets are broken the nucleic acids (labeled with barcoding oligonucleotides) may be pooled or combined together, amplified, sequenced, etc. Because nucleic acids from different droplets are tagged with barcoding oligonucleotides the nucleic acid molecules can be computationally deconvoluted.

In one set of embodiments, the microfluidics chip is manufactured from an elastomeric polymer such as polydimethylsiloxane (“PDMS”), polytetrafluoroethylene (“PTFE” or Teflon®), or the like [32]. Other examples of potentially suitable polymers include, but are not limited to, polyethylene terephthalate (PET), polyacrylate, polymethacrylate, polycarbonate, polystyrene, polyethylene, polypropylene, polyvinylchloride, cyclic olefin copolymer (COC), polytetrafluoroethylene, a fluorinated polymer, a silicone such as polydimethylsiloxane, polyvinylidene chloride, bis-benzocyclobutene (“BCB”), a polyimide, a fluorinated derivative of a polyimide, or the like. The device may also be formed from composite materials, for example, a composite of a polymer and a semiconductor material. In a non-limiting example, the PDMS polymer is used which is sold under the trademark Sylgard by Dow Chemical Co., Midland, MI, and particularly Sylgard 182, Sylgard 184, and Sylgard 186.

In a still further aspect, the present invention may provide kits for use in performing the methods described herein. The kits may comprise the reagents and precursors necessary to put the methods into effect, including the particles and microcapsules described herein, or alternatively may include the particles and the precursors necessary to form the microcapsules around the biological species. In addition, the kits may include one or more microfluidic chips for making the microcapsule and/or making the droplet, as well as microfluidic consumables and appropriate reagents.

### Microcapsules

As described above, the microcapsules to be used in the methods of the present invention have a semi-permeable shell and a core. The semi-permeable shell of the microcapsule retains the cell (or other biological species) and the nucleic acids released therefrom inside the microcapsule while allowing smaller molecular weight compounds to diffuse into and out of the core of the microcapsule.

In general, the semi-permeable shell allows for the diffusion of smaller molecular weight compounds of approximately MW 200,000 or less through the shell, while retaining larger molecular weight compounds of approximately MW 300,000 and above. Similarly, the shell may be permeable to compound of less than 120,000±80,000 Da.

In general, the microcapsules have high circularity and high concentricity. Considering the average radius, R, of the microcapsule being R=V (square root over (S/Tt)), wherein S is the equatorial transverse surface of the capsule. The circularity, C, is a ratio of the minor axis (R min) over the major axis (R max) of the ellipse adjusted to the external edge of the projected equatorial section. In the present disclosure the microcapsule may have a circularity C=0.8±0.2, preferably C=0.9±0.1, more preferably C=0.94±0.06, and even more preferably C=0.95±0.05.

The concentricity, O, of the microcapsule is defined as O=(Wmin/Wmax)*100%, wherein Wmin is thinnest part of the shell and Wmax is the thickest part of the shell. In the present disclosure the microcapsule shows O≥66%. The high circularity and concentricity of microcapsules may be advantageous during the performance of reactions in the microcapsule to ensure that reactions are efficient.

The semi-permeable shell of the microcapsule may comprise a gel formed from a polymer, wherein the polymer in the gel is covalently cross-linked. In particular, the polymer is a polyampholyte and/or a polyelectrolyte or a synthetic polymer. The term ‘polyampholyte’ refers to a polyelectrolyte that bears both cationic and anionic groups, or corresponding ionizable groups, and where the ‘polyelectrolytes’ are polymers whose repeating units bear an electrolyte group. It should be understood that term ‘polyampholyte’ and ‘ampholytic polymer’ are synonyms as defined by IUPAC.

In particular, the gel may be formed from a polyampholyte and/or a polyelectrolyte that comprises a covalently cross-linkable group, or may be formed from a polyampholyte and/or a polyelectrolyte or synthetic polymer that is modified with a chemical group, which chemical group participates in the covalent cross-link. The chemical group may be selected from the group consisting of acrydite, acrylate, methacryloyl, acrylamide, methacrylamide, bisacrylamide, methacrylate, methacrylic acid, acrylic acid, polyacrylic acid, methacrylic anhydride, acryloyl, vinyl, vinylsulfone, vinylpyrrolidone, thiol, disulphide, cystamine, carboxyl, amine, imine, azide, triazole, tetrazine, azidophenylalanine, alkynyl, alkenyl, alkynes, diisocyanate, hydroxypropionic acid, hydroxy phenol, azobenzene, methylcyclopropene, trans-cyclooctene (TCO), norbornene, diacrylcyclooctyne (DBCO) or cyclooctanyl moieties and/or reagents.

The polymer may comprise a protein, peptides, oligopeptides or polypeptides, or any combination thereof. Accordingly, the polymer may be described as “proteinaceous”. In particular, where the polyampholyte is a protein, polypeptides or oligopeptide, the primary amino acid sequence may comprise at least 10% disorder promoting amino acids, and preferably at least 30%. Disorder promoting amino acids include proline, glycine, glutamic acid/glutamate, serine, lysine, alanine, arginine, and glutamine. Without wishing to be bound by theory it is considered that such disorder promoting amino acids also promote liquid-liquid phase separation in the droplet during formation of the microcapsule (which is discussed further below). Furthermore, the proteinaceous polyampholytes that show liquid-liquid phase separation properties are often characterized by long segments of low diversity amino acids. These segments are often repetitive and are enriched in glycine (G), glutamine (Q), asparagine (N), serine(S), arginine (R), lysine (K), aspartate (D), glutamate (E) or aromatic amino acids such as phenylalanine (F) and tyrosine (Y) amino acids. These segments often encompass multiple short motifs such as YG/S-, FG-, RG-, GY-, KSPEA-, SY- and Q/N-rich regions, or regions of alternating charges [33].

The polyampholyte may be one that is capable of forming a coacervate in response to salts, temperature change, pH change or ionic change of a solvent in which the said polyampholyte is present during formation of the semi-permeable shell.

In preferred examples the polyampholyte and/or the polyelectrolyte is a “thermo-responsive” polymer capable of forming a gel in response to a temperature change, for example when cooled, below sol-gel transition temperature. The gel that is formed in response to the temperature change is a mesh or 3-dimensional network of polymer strands, with a solid structure due to physical cross-linking of individual polymer strands.

For example, the polyampholyte may be selected from the group consisting of collagen, mucin, laminin, elastin, elastin-like polypeptides, fibrin, silk fibrion, fibronectin, vimentin, glycinin, gluten, casein, or hydrolyzed forms thereof, such as gelatin.

Particularly preferred is a polyampholyte is selected from gelatin, gelatin methacryloyl, gelatin methacrylamide, gelatin acrylamide and gelatin methacrylate, and preferably is gelatin methyacrylate.

The core of the microcapsule may comprise an antichaotropic agent and/or a polyhydroxy compound. In particular, the The antichaotropic agent may be kosmotropic salt, and in particular may be a carbonate, a sulphate, a phosphate or a citrate. In a preferred example kosmotropic salt is an ammonium sulphate.

The polyhydroxy compound may be a synthetic polymer or a naturally occurring polymer or derivatives thereof. In particular, the polyhydroxy compound may be selected from a polysaccharide, a carbohydrate, an oligosaccharide, or a sugar, which can be natural or synthetic. In one example, the polyhydroxy compound is one or more of dextran, alginate, hyaluronic acid, glucan, glycogen, starch (amylose, amylopectin), agarose, agar-agar, heparin, pectin, cellulose (including hydroxyethyl cellulose), hemicellulose, chitosan, chitin, xanthan gum, curdian, pullulan, inulin, graminan, levan, carrageenan, polyglycerol, and derivatives of the foregoing that are chemically modified or partly hydrolyzed. Preferably the polyhydroxy compound is glucan, more preferably dextran. Alternatively, the polyhydroxy compound may be a synthetic polymer, such as Ficoll (e.g. Ficoll PM 4000).

The polyhydroxy compound may have a molecular weight of 300 Da to 5000 kDa. In one example the molecular weight is greater than 10 kDa (i.e. is between 10 kDa and 800 kDa). In another example the molecular weight is greater than 100 kDa (i.e. is between 100 kDa and 800 kDa). In a preferred example, the polyhydroxy compound has a molecular weight of 400 to 600 kDa, more preferably approximately 500 kDa.

The method of forming a microcapsule around a biological species, e.g. a cell, may comprise:
(1) forming water-in-oil droplets comprising a biological species, a first solute and a second solute, wherein the first solute comprises a polyampholyte and/or a polyelectrolyte comprising one or more covalently cross-linkable groups,
(2) allowing aqueous phase separation inside the water-in-oil droplet into a shell phase enriched in the first solute and a core phase enriched in the second solute, and gelation and/or precipitation in the shell phase to form an intermediate microcapsule;
(3) forming intermolecular covalent cross-links with the one or more covalently cross-linkable groups to form the microcapsule comprising a semi-permeable shell of covalently cross-linked polymer and a core, wherein the cell is in the core of the microcapsule. In particular, the second solute may be the antichaotropic agent and/or a polyhydroxy compound.

In preferred cases, the polymer is a thermo-responsive polymer as described above. In such cases, step (2) may comprise changing the temperature of the water-in-oil droplet so as to induce physical gelation of the thermo-responsive polymer to achieve solidification in the shell phase to form the intermediate microcapsule, wherein the solidified gel is a thermoreversible gel. Changing the temperature may comprises cooling the water-in-oil droplet to a temperature between 4° C. and 30° C., and preferably to below 10° C.

The covalent cross-linking in (3) may comprise exposing the intermediate microcapsule to a chemical agent, irradiation, or heat, or any combination thereof, to covalently cross-link the polymer. Preferably, (3) comprises covalently cross-linking by photo-polymerisation.

An example of the performance of the above method is set out below in Example 1.

The following are intended as examples only and do not limit the present disclosure.

MATERIALS:

| | Cat. No. |
|---|---|
| Microcapsule production | |
| Gelatin methacryloyl | Sigma-Aldrich, 900496-1G |
| Dextran (MW 500K) | Sigma-Aldrich, 31392-10G |
| Lithium phenyl-2,4,6-trimethylbenzoylphosphinate (LAP) | Sigma-Aldrich, 900889-1G |
| 10X PBS | Gibco, 70011044 |
| 10X DPBS | Gibco, 14190144 |
| Pluronic F-68 | Gibco, 24040032 |
| Droplet stabilization oil | Droplet Genomics, DG-DSO-20 |
| Emulsion breaker | Droplet Genomics, DG-EB-1 |
| Light mineral oil | Sigma-Aldrich, M5904 |
| Bacteria lysis | |
| Ready-Lyse Lysozyme Solution | Lucigen, R1810M |
| GeneJET RNA Purification Kit/Lysis Buffer | Thermo Scientific, K0732 |
| 500 mM EDTA, pH [8.0] | Invitrogen, 15575020 |
| DTT | Thermo Scientific, R0861 |
| NaCl | Sigma-Aldrich, S9888 |
| 1 M Tris-HCl, pH [7.5] | Invitrogen, 15567027 |
| Triton X-100 | Thermo Scientific, 85111 |
| gDNA depletion | |
| RapidOUT DNA Removal Kit | Thermo Scientific, K2981 |
| Polyadenylation | |
| E. coli Poly(A) Polymerase (includes buffer and ATP) | NEB, M0276 |
| cDNA synthesis (template switching) | |
| Barcoded Hydrogel Beads | — |
| dNTPs, 10 mM each | Invitrogen, R0192 |
| 40 U/μL RiboLock RI | Thermo Scientific, EO0381 |
| 200 U/μL Maxima H Minus RT and Buffer | Thermo Scientific, EP0751 |
| TSO primer (HPLC purified): | IDT |
| 5′-AAGCAGTGGTATCAACGCAGAGTACATrGrGrG-3′ | |
| Igepal CA-630 | Sigma-Aldrich, 18896 |
| Nucleic acid purification/Capsule decomposition | |
| AMPure magnetic beads | Beckman Coulter, A63880 |
| Dextranase | Sigma-Aldrich, D0443-50ML |
| Proteinase K solution, 20mg/mL | Invitrogen, AM2548 |
| Collagenase A | Roche, 10103586001 |
| cDNA amplification and library preparation | |
| KAPA HiFi HotStart ReadyMix (2X) | KAPA, KK2602 |
| P5 primer: 5′-TACGGCGACCACCGAGATC-3′ (SEQ ID 1) | IDT |
| TSO primer: 5′-AAGCAGTGGTATCAACGCAGAG-3′ (SEQ ID 2) | IDT |
| NEBNext® UltraTM II FS DNA Library | NEB, E7805S |
| Prep Kit for Illumina | |
| Ligation adapter: | |
| /5Phos/GATCGGAAGAGCACACGTCTGAACTCCAGTCAC/3ddC | IDT |
| (SEQ ID 3) | |
| /5AmMC6/GCTCTTCCGATCT (SEQ ID 4) | IDT |
| Agilent BioAnalyzer HS DNA chip and reagents | Agilent, 5067-4626 |

Capsule recovery and cell washing buffer: 1x DPBS with 0.1% F-68
Capsule washing buffer: 10 mM Tris-HC1, pH [7.5] with 0.1% Triton X-100
Lysis buffer 1: 10 mM Tris-HCl, pH [7.5], 100 mM NaCl, 1 mM EDTA, 0.1% Triton X-100 and 50 U/μL Lysozyme
Lysis buffer 2: GeneJET RNA Purification Lysis Buffer with 40 mM DTT

### Example 1

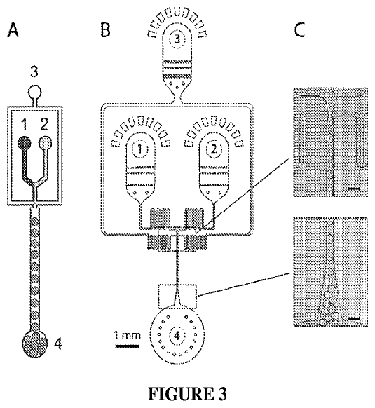

This example makes use of SPMs for encapsulating mammalian cells, bacteria and other biological species. The individual cells are isolated in microfluidic droplets composed of aqueous two-phase system (ATPS) [34]. One non-limiting embodiment of such ATPS is gelatin methacrylate (GMA) and 500K dextran blend (FIG. 3). Once this blend is encapsulated within microfluidic droplets the phase separation occurs and GMA forms a shell, and dextran forms a core. Upon polymerization the GMA may be solidified into chemically cross-linked hydrogel, while core will remain in a liquid form or a semi-liquid.

As a model system the K562 (ATCC, CCL-243) and NIH/3T3 (ATCC, CRL-1658) cells were isolated in microfluidic droplets using a microfluidics chip 40 μm height and having a nozzle 40 μm wide. The typical flow-rates for introducing fluids to microfluidics system are in the range of 50-2000 μl/h gelatin methacrylate solution (Sigma-Aldrich, 900496-1G); 50-2000 μl/h for dextran solution (Sigma-Aldrich, 31392-10G) with cells and 100-5000 μl/h for the carrier oil. In a specific example the flow rates were 250 μl/h gelatin methacrylate solution (Sigma-Aldrich, 31392-10G); 100 μl/h for dextran solution (Sigma-Aldrich, 31392-10G) with cells and 700 μl/h for the carrier oil (Droplet Genomics, DG-DSO-20). The cells were suspended in dextran solution prior the encapsulation.

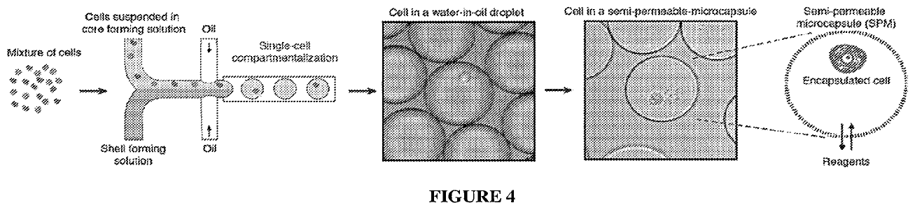

As stated above the liquid-liquid phase separation inside ATPS droplets results in dextran-rich core and GMA-rich shell. In non-limiting example, the SPMs were generated by incubating ATPS droplets at selected temperature to induce the sol-gel transition and solidification of the GMA-rich shell. Next, the resulting solidified SPMs were released from the emulsion, re-suspended in an aqueous buffer containing photo-initiator and photo-illuminated to induce chemical cross-linking of a shell. In a more specific embodiment, the ATPS droplets were incubated at ˜4° C. for 30-60 min. to induce temperature-responsive gelation of GMA-rich phase. Continuing procedure on ice, the SPMs were recovered from the emulsion using commercial emulsion breaker (Droplet Genomics, DG-EB-1) and released into 1×PBS buffer (Gibco, 70011044) supplemented with 0.1% Pluronic F-68 (Gibco, 24040032). The suspension having SPMs was transferred to a new 1.5 ml tube, supplemented with 0.1% (w/v) lithium phenyl-2,4,6-trimethylbenzoylphosphinate (LAP) (Sigma-Aldrich, 900889-1G) and photo-polymerized under exposure to 405 nm LED device (Droplet Genomics, DG-BR-405) for 20 secs. Following the aforementioned two-step polymerization procedure, the SPMs contained a clear, well-centered core enriched in dextran, and a solidified hydrogel shell composed of covalently cross-linked gelatin (FIG. 4).

In this particular example, cell encapsulation was performed on a microfluidic device prepared by soft-lithography, but emulsification can be also performed using other tools such as capillaries or tubing, for example. Other microfluidic configurations can also be used. In one set of embodiments, the size of the ATPS droplets was approximately 70 μm but it could be readily adjusted using different flow rates or a microfluidics chip having different microchannels. The microfluidic device used in this example has one inlet for droplet carrier oil, two inlets for aqueous phases. One aqueous inlet is used for injecting cell suspension and a second aqueous inlet is used for injecting the shell forming precursors (e.g. GMA). For the carrier oil, fluorinated oil (e.g. HFE-7500) containing ˜1.5% (v/v) surfactant (PFPE-PEG-PFPE tri-block copolymer containing two perfluoropolyether blocks (PFPE) and one poly(ethylene) glycol (PEG) block) was used. The carrier oil used for emulsification is not limited to fluorinated liquids and alternative fluids such as based on hydrocarbons (e.g. mineral oil, hexane, etc.), silicon oil and other type of oils can be employed successfully.

The cell suspension was prepared in this example with the following considerations. The cell number density (cells per unit volume) was adjusted to minimize incidences of two or more cells becoming captured in the same droplet. The precise calculation of the correct number density depends, for example, on factors such as the amount of multi-cell events that can be tolerated, and on the droplet volume, and on the relative droplet volume contributed by the cell suspension. For example, a cell suspension containing 0.1 million cells, was transferred into a new 1.5 ml tube, pelleted, re-suspended in 100 ul of 15% (w/v) dextran (Sigma-Aldrich, 31392-10G) and used immediately for the encapsulation.

### Example 2

This example illustrates generation of SPMs with single-cells, lysing the cells to generate the SPMs carrying single-cell lysates, and genomic DNA depletion of a single-cell lysates within the SPMs. It should be understood that once cells are encapsulated they can be lysed using different conditions, for example, using ionic or non-ionic detergents, denaturating agents (e.g., guanidinium chloride, urea, etc.), chaotropic agents, enzymes (e.g. lipases, lysozyme, etc.) and other lytic agents. In this example lysis of encapsulated cells was performed by suspending SPMs containing encapsulated cells in GeneJET RNA Purification Kit Lysis Buffer (Thermo Scientific, K0732) supplemented with 40 mM DTT (Thermo Scientific, R0861). After initial lysis, SPMs were re-suspended in a fresh lysis buffer and incubated at room temperature for additional 5 minutes and rinsed in a fresh lysis buffer. After lysis, the resulting single-cell lysates within the SPMs was rinsed five-times in a washing buffer (10 mM Tris-HCl [pH 7.5] (Invitrogen, 15567027), 0.1% (v/v) Triton X-100 (Thermo Scientific, 85111)). During these procedures the centrifugation steps were performed at 2000 g for 2 minutes at 4° C.

To digest the genomic DNA 100 μl of close-packed SPMs were mixed with 100 μl solution containing 0.1/ul DNase I (Thermo Scientific, K2981), 2×DNAse reaction buffer (Thermo Scientific, K2981) and 0.4 U/ul RiboLock RNase Inhibitor (Thermo Scientific, EO0381), and incubated at 37° C. for 20 minutes. Next, additional 5 U of DNase I (Thermo Scientific, K2981) were added to reaction mix and incubated for additional 10 minutes at 37° C. Finally, the SPMs were rinsed three-times in washing buffer (10 mM Tris-HCl [pH 7.5] (Invitrogen, 15567027), 0.1% (v/v) Triton X-100 (Thermo Scientific, 85111)) and were used for nucleic acid (e.g., mRNA) barcoding as detailed below. In this context it should be understood that DNAse I can be replaced with other enzymatic treatments. For example, applications that require DNA barcoding would replace DNAse I treatment with other enzymatic reaction(s) such as transposition, restriction endonuclease hydrolysis and other enzymes that do not degrade chromosome down to single-, di or tri-nucleotides. Similarly, the applications that rely on nucleic acid modification would replace DNAse I with other enzymatic reactions such as polyadenylation, G-capping, phosphorylation, dephosphorylation, adenylation, ligation, nucleic acid base modification, etc. Those of ordinary skill in the art will be aware of techniques for preparing nucleic acids for further analysis and sequencing.

### Example 3

This example illustrates certain techniques for barcoding nucleic acids.

These examples are better understood together with FIGS. 2, 5 and 6. In an exemplary embodiment, the SPMs carrying lysate of individual cells are introduced into a microfluidics device and are co-encapsulated along with hydrogel beads and assay reagents within microfluidic droplets. The hydrogel bead carries barcoding oligonucleotides covalently attached thereto. Once encapsulated the barcoding oligonucleotides are released by melting the hydrogel bead in the presence of chemical agent (e.g. reducing agent DTT). Alternatively, the barcoding oligonucleotides can be released by other means such as photo-illumination or enzymatic-cleavage. The nucleic acid molecules are attached to the barcoding oligonucleotide tags through the enzymatic (e.g. ligation, primer extension, PCR) or chemical (e.g. click-chemistry) reaction. It should be understood that certain applications may rely on nucleic molecule capture and barcoding without releasing barcoding oligonucleotides from the beads. For example, nucleic acid molecules of lysed cell could be captured on a bead within a droplet, as has been shown previously [35].

The SPMs suspended in aqueous buffer can be packed (concentrated) such that their delivery into droplets becomes ordered and synchronized, ensuring that the majority of droplets host a desirable number of SPMs, for example, exactly one SPM. Likewise, the hydrogel beads carrying barcoding DNA primers suspended in aqueous buffer can also be packed (concentrated) such that their delivery into droplets becomes ordered and synchronized, ensuring that the majority of droplets host exactly one bead (FIG. 5). As a result, the co-delivery of a SPM and a bead into the same droplet can be precisely controlled and high co-occupancy events (one SPM and one bead) can be achieved. In a typical scenario over 50% of droplets will contain one SPM and one bead.

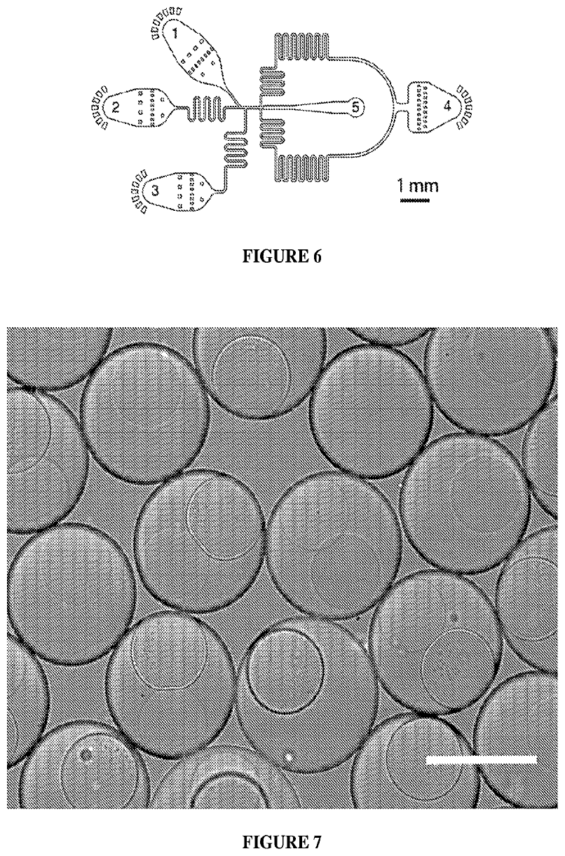

As one non-limiting example, the fluids are delivered into the microfluidics device FIG. 6. The device has four inlets and one outlet. The inlets are used to introduce i) SPMs, ii) hydrogel beads, iii) biological and/or chemical reagents and iv) carrier oil. The SPMs, hydrogel beads, and assay reagents can be introduced into a microfluidics chip through either inlet. Droplet generation occurs at or downstream the flow-focusing junction and co-encapsulated SPMs and hydrogel beads are then collected at the outlet. The flow rate of each inlet can be adjusted in order to obtain optimal conditions for one SPM and one hydrogel bead co-encapsulation events. In a particular embodiment the flow rates in the range of 100 microliters/hour (ul/h), 100 ul/h, 250 ul/h and 400 ul/h, are applied for the SPMs, hydrogel beads, reaction mix, and carrier oil with surfactant, respectively. However, the flow rates of all phases can be adjusted independently between 1 and 10,000 ul/h, depending on the particular application.

Once encapsulated, the nuclei acid molecules inside the SPMs can be released by breaking the SPM shell using enzymatic or chemical means. Alternatively, the nuclei acid molecules inside the SPMs can be processed further without breaking the SPM. Irrespectively whether the SPMs are broken or not, the mRNA molecules can be converted to cDNA using reverse transcription (RT). During the RT step the barcoding nucleotides anneal to poly(A) part of mRNA molecules via 3′ end poly(T) tail, and get extended to cDNA. In another non-limiting example, DNA fragments inside the SPMs can be ligated to barcoding DNA oligonucleotides. In yet another non-limiting example, the barcoding DNA oligonucleotides can tag the nucleic acid fragments by primer extension reaction, or PCR.

In one particular example the reverse transcription (RT) reaction mix with and without collagenase was prepared as indicated in Table 1. 150 μl of RT reaction mix was infused into a microfluidics chip (FIGS. 5 and 6) along with SPMs and hydrogel beads in order to perform nucleic acid barcoding.

TABLE 1
RT reaction mix composition.

| | Volume, | |
|---|---|---|
| Reagent | μl | Concentration |
| 5X RT buffer | 30 | 2X |
| dNTPs, 10mM each | 15 | 1 mM each |
| TSO primer, 0.5 mM (SEQ ID 5) | 7.5 | 25 μM |
| 5′-AAG CAG TGG TAT CAA | | |
| CGC AGA GTA CAT rGrGrG-3′ | | |
| Igepal CA-630, 10% | 9 | 0.6% |
| Maxima H minus, 200 U/μl | 15 | 20 U/μl |
| Ribolock, 40 U/μl | 15 | 4 U/ul |
| Collagenase, 20 mg/ul | 6 | 0.8 mg/μl |
| Water | Up | |
| | to | |
| | 150 | |

Droplets carrying co-encapsulated SPMs, hydrogel beads and assay reagents may be collected off-chip and retain integrity (FIG. 7). Droplet collected off-chip can be incubated at desirable temperature for an extended period of time. For example, collected droplets can be incubated at 50° C. for 60 min followed by 15 min incubation at 85° C. in order to perform RT reaction.

To release the barcoded nucleic acid (DNA or RNA) molecules droplets and SPMs can be broken by chemical or physical techniques. Typically, the emulsion droplets are broken using perfluoro-octanol. The water-in-oil droplets in this particular example were broken by adding 10% perfluoro-octanol (Sigma-Aldrich, 370533) onto the collected emulsion. As one non-limiting example, 1/50^{th} volume of dextranase (Sigma, D0443-50ML) and 1/50^{th} volume of collagenase A (Roche, 10103586001) was added to the broken emulsion and incubated at 37° C. for 10 min in order to decompose the SPMs. It should be understood that the decomposition (breakup) of SPMs will depend on their chemical composition and can rely on chemical (e.g. use of alkaline solution) or enzymatic (e.g. use of hydrolase) treatment. In one example revealed here the SPMs are broken by using collagenase A enzyme (Roche, 10103586001).

The barcoded nucleic acid molecules present in aqueous phase of a broken emulsion can be amplified or further processed for sequencing. In one non-limiting example revealed here the aqueous phase of broken emulsion was spun down through Zymo Spin-IC column, the flow-through fraction was collected and purified 2-times with 0.8× AMPure magnetic beads (Beckman Coulter, A63880), and eluted in 20 μl of water.

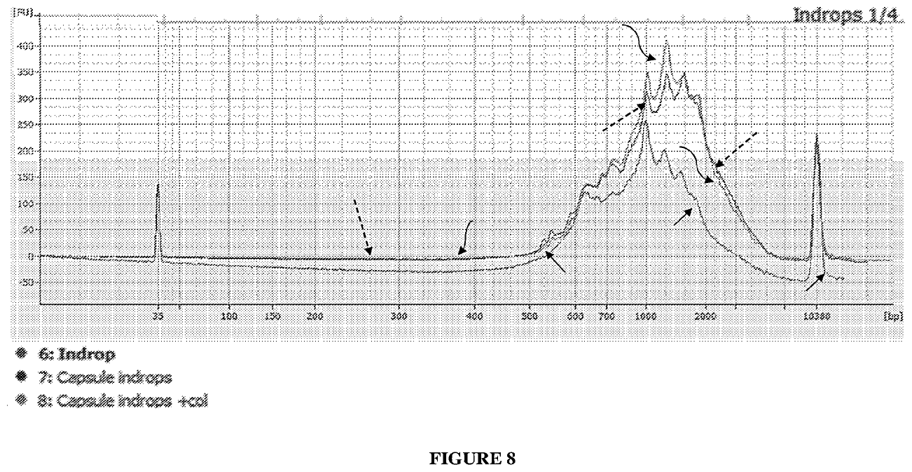

The barcoded-cDNA may be amplified by PCR. For example, FIG. 8 shows barcoded-cDNA released from droplets and amplified by 13-cycles of PCR using Kapa HiFi 2× Ready Mix (KAPA, KK2602). Tables 2 and 3 indicate the PCR reaction mix and cycling conditions for amplifying the barcoded cDNA.

TABLE 2
cDNA amplification mix

| | Volume, | Concentration |
|---|---|---|
| Reagent | μl | in 2X mix |
| 2X KAPA HiFi ReadyMix | 25 | 1X |
| PCR primer mix*, | 5 | 0.5 uM each |
| 5 μM each | | |
| Purified cDNA | 20 | |
| Total | 50 | |

*PCR primer mix
P5 primer 5′-TACGGCGACCACCGAGATC-3′ (SEQ ID 1)
TSO primer 5′-AAGCAGTGGTATCAACGCAGAG-3′ (SEQ ID 2)

TABLE 3
PCR program settings

| Step | Temperature | Time |
|---|---|---|
| Initial denaturation | 98° C. | 00:03:00 |
| Denaturation | 98° C. | 00:00:15 |
| Annealing | 67° C. | 00:00:20 |
| Extension | 72° C. | 00:01:00 |

Go to step 2, 12 cycles (13 in total)

| | | |
|---|---|---|
| Final extension | 72° C. | 00:01:00 |
| Hold | 4° C. | Hold |

The amplified cDNA can be further processed to construct sequencing library. For example, the barcoded-cDNA amplified by PCR was purified 2-times with 0.6× AMPure magnetic beads, eluted into 20 μl of water and fragmented as follows (Table 4):

TABLE 4
DNA fragmentation reaction composition

| Component | Volume, μL |
|---|---|
| Amplified cDNA (6-9 ng/μL) | 5 |
| Water | 10.25 |
| NEBNext Ultra FS Reaction buffer (vortex before use) | 1.75 |
| NEBNext Ultra FS Enzyme mix (vortex before use) | 0.5 |
| Total: | 17.5 |

Samples were vortexed and spin-down and DNA fragmentation was carried out at 37° C. for 8 min, followed by 30 min at 65° C. After reaction double size selection (0.6×-0.8×SPRI) was performed. Purified fragmented DNA was eluted in 17.5 μL of water and ligated to adapter using following reaction mix (Table 5):

TABLE 5
Reaction composition for
ligation of PCR adapter

| Component | Volume, μL |
|---|---|
| Fragmented DNA | 17.5 |
| NEBNext Ultra II | 7.5 |
| Ligation Master Mix | |
| NEBNext Ultra Ligation | 0.25 |
| Master Enhancer | |
| Ligation adapter * | 1.25 |
| Water | 7.75 |
| Total: | 34.25 |

* Ligation adapter is a duplex DNA having two oligonucleotides:
5′-/5Phos/GATCGGAAGAGCACACGTCTGAACTCCAGTCAC/3ddC-3′ (SEQ ID 3)
5′-/5AmMC6/GCTCTTCCGATCT-3′ (SEQ ID 4)

Ligation was carried out at 20° C. for 15 min (lid at 30° C.). After reaction total volume was brought to 100 μL, 0.8× ampure was performed and ligation product was eluted in 40 μL.

Next indexing PCR was conducted by preparing the PCR reaction mix and cycling conditions indicated in Table 6 and 7 below:

TABLE 6
PCR reaction composition

| Component | Volume, μL |
|---|---|
| 2x Kappa | 25 |
| Template from previous step | 20 |
| Indexing primers* (5 μM each) | 5 |
| Total: | 50 |

*Indexing primers
P5 indexing primer: 5′-AATGATACGGCGACCACCGAGATCTACAC (SEQ ID 6)
P7 index 6: 5′-CAAGCAGAAGACGGCATACGAGATCAGTGGGTGACTGGAGTTCAGACGTG*T (SEQ ID 7)
P7 index7: 5′-CAAGCAGAAGACGGCATACGAGATGTTGTCGTGACTGGAGTTCAGACGTG*T (SEQ ID 8)
P7 index 8: 5′-CAAGCAGAAGACGGCATACGAGATTGACAAGTGACTGGAGTTCAGACGTG*T (SEQ ID 9)

TABLE 7
PCR cycling conditions

| Step | Temperature | Duration | | Cycles |
|---|---|---|---|---|
| Initial denaturation | 95° C. | 3 | min | 1 |
| Denaturation | 98° C. | 20 | s | 11 |
| Annealing | 54° C. | 30 | s | |
| Extension | 72° C. | 20 | s | |
| Final extension | 72° C. | 1 | min | 1 |

| | | |
|---|---|---|
| Hold | 4° C. | Hold |

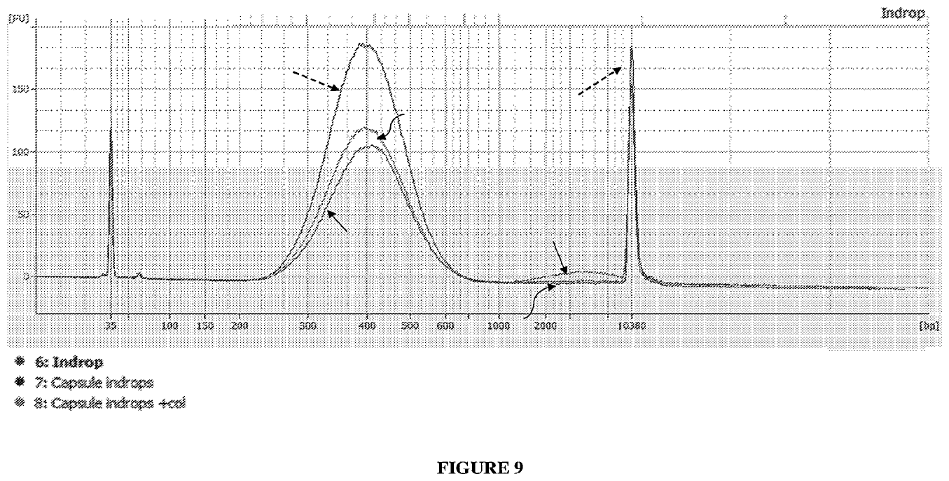

The amplified DNA library was purified using double size selection (0.6-0.8× AMPure magnetic beads (Beckman Coulter, A63880)) and eluted in 15 μL. The DNA library quality was then verified on Agilent BioAnalyzer HS DNA chip (Agilent, 5067-4626), as shown in FIG. 9.

Once DNA library is generated the primary sequence of nucleic acids, including the barcode and unique molecular identifiers can be determined by sequencing. It should be understood that the above example can be used to prepare and analyze, as non-limiting examples, genomes, transcriptomes, epigenomes, single nucleotide polymorphisms, specific gene expression levels, non-coding RNA, entire genes or their sections, etc.

### Example 4

This example illustrates certain techniques for preserving the SPMs carrying cell lysate including nucleic acids in alcohol. In a non-binding example, the Escherichia coli cells having optical density O.D.˜2.0, and Bacillus subtilis cells having optical density O.D.˜2.0, were resuspended in 1×DPBS (Gibco, 14190144) buffer and 2.5 ul of E. coli bacteria suspension and 2.5 ul of B. subtilis bacteria suspension were combine with 95 ul of 15% Dextran (MW 500K) (Sigma-Aldrich, 31392-10G). Using 20 μm deep microfluidics device (FIG. 3) the cells were encapsulated in ATPS droplets. The following flow rates were used: 3% (w/v) gelatin methacrylate solution (Sigma-Aldrich, 900496-1G)—50 μL/h, 15% dextran solution carrying bacteria cells—20 μL/h and droplet stabilization oil (Droplet Genomics, DG-DSO-20)-400 μl/hr. ATPS droplets were collected off-chip and incubated at 4° C. for 40 minutes to induce the solidification of the shell. Continuing procedure on ice, the SPMs were recovered from the emulsion using commercial emulsion breaker (Droplet Genomics, DG-EB-1) and released into 1×DPBS buffer (Gibco, 14190144) supplemented with 0.1% Pluronic F-68 (Gibco, 24040032). The suspension having SPMs was transferred to a new 1.5 ml tube, supplemented with 0.1% (w/v) lithium phenyl-2,4,6-trimethylbenzoylphosphinate (LAP) (Sigma-Aldrich, 900889-1G) and photo-polymerized under exposure to 405 nm light emitting diode (LED) device (Droplet Genomics, DG-BR-405) for 20 seconds. The SPMs were washed twice in 1×DPBS (Gibco, 14190144) with 0.1% Pluronic F-68 (Gibco, 24040032) before proceeding to the next operation.

To lyse encapsulated cells the SPMs were dispersed in 1 mL buffer (10 mM Tris-HCl, pH [7.5] (Invitrogen, 15567027), 100 mM NaCl (Sigma-Aldrich, S9888), 1 mM EDTA (Invitrogen, 15575020), 0.1% Triton X-100 (Thermo Scientific, 85111), sedimented at 1000 g for 2 minutes, and supernatant replaced with lysis buffer (10 mM Tris-HCl, pH [7.5] (Invitrogen, 15567027), 100 mM NaCl (Sigma-Aldrich, S9888), 1 mM EDTA (Invitrogen, 15575020), 0.1% Triton X-100 (Thermo Scientific, 85111) and 50 U/μL Lysozyme (Lucigen, R1810M). After incubation at room temperature for 15 minutes, the SPMs were spun down at 1000 g for 2 minutes and the supernatant replaced with fresh lysis buffer. Following 5 min incubation at room temperature, the capsules were spun down again, and washed 4-5 times with washing buffer (10 mM Tris-HCl, pH [7.5] (Invitrogen, 15567027) and 0.1% Triton X-100 (Thermo Scientific, 85111)). Finally, SPMs were resuspend in 70% ice-cold ethanol and stored at −20° C.

### Example 5

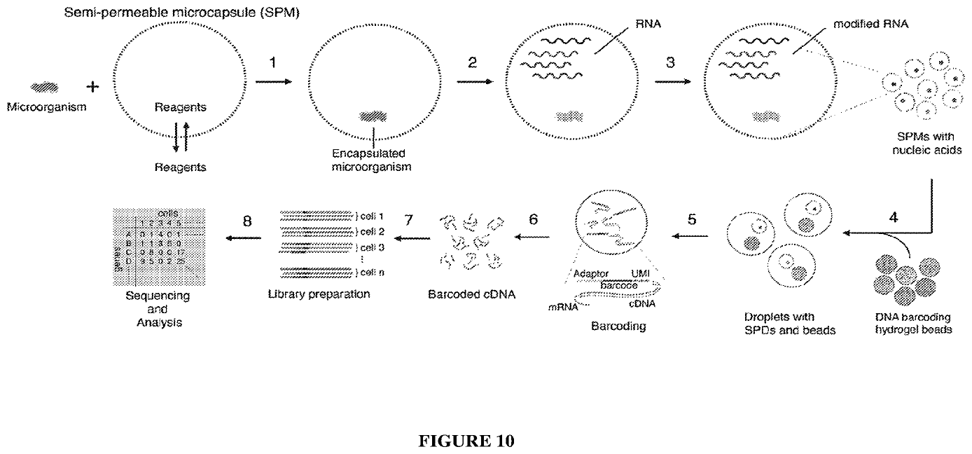

This example illustrates certain techniques for modifying the nucleic acids derived from single cells followed by the barcoding of said modified nucleic acids. The overall strategy of this particular example is best understood along with FIG. 10. As described above the individual cells, e.g., eukaryotic and prokaryotic, may be isolated in SPMs, lysed and preserved in alcohol suspension for extended periods of time. Once the SPMs with single-cell lysate are generated the nucleic acids can be further processed, or modified as required for a particular assay. In a non-binding example, the SPMs carrying cell lysates of individual E. coli and B. subtilis bacteria cells were treated with DNAse I (Thermo Scientific, K2981) enzyme to deplete the genomic DNA followed by RNA modification. In this particular example, the RNA modification is polyadenylation of RNA. The SPMs carrying lysed cells including nucleic acids, and preserved in 70% ethanol, were washed three times in a washing buffer (10 mM Tris-HCl, pH [7.5] (Invitrogen, 15567027) and 0.1% Triton X-100 (Thermo Scientific, 85111)) and combined with gDNA depletion reagents provided in RapidOUT DNA Removal Kit (Thermo Scientific, K2981). Specifically, 1× DNase I buffer with MgCl₂ (Thermo Scientific, K2981), 0.05 U/L DNase I (Thermo Scientific, K2981), 0.4 U/μL RiboLock RI (Thermo Scientific, EO0381) was used to treat SPMs at 37° C. for 20 minutes. After said incubation, another 5 U of DNase I enzyme (Thermo Scientific, EO0381) were added to reaction mix and incubated for additional 10 min. The DNA depletion reaction was terminated by washing SPMs 4-5 times in a washing buffer.

To showcase a non-binding example of nucleic acid modification reaction the SPMs having cell lysates depleted of genomic DNA were subjected to polyadenylation reaction. The reaction mix containing SPMs dispersed in 1× E. coli Poly(A) Polymerase Reaction Buffer (NEB, M0276), 1 mM ATP (NEB, M0276), 0.4 U/μL E. coli Poly(A) Polymerase (NEB, M0276) were incubated at 37° C. for 30 minutes. Following the incubation, the SPMs were washed 2-3 times in a washing buffer, strained through 40 μm size strainer and close-packed by centrifugation at 2000-5000 g for 5 minutes. The SPMs carrying modified nucleic acids were then loaded into a microfluidics chip with nucleic acid barcoding reagents as described below.

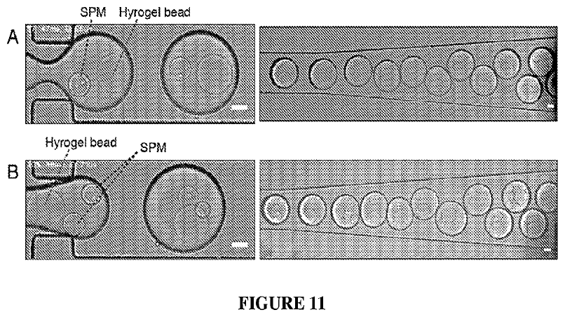

In an exemplary embodiment, the SPMs carrying modified nucleic acids were introduced into a microfluidics device and are co-encapsulated along with hydrogel beads and assay reagents within microfluidic droplets. The hydrogel bead carried covalently attached barcoding DNA oligonucleotides and assay reagents included the reverse transcription (RT) reaction mix. The SPMs, hydrogel beads and assay reagents were delivered into the microfluidics device shown on FIG. 6. The microfluidics device contains four inlets and one outlet. The inlets are used to introduce i) SPMs, ii) hydrogel beads, iii) RT reaction mix and iv) carrier oil. The flow rate of each inlet can be adjusted so that most of the microfluidics droplets end up having one SPM and one hydrogel bead (FIG. 11A). However, it is important to emphasize that adjusting the flow rates one can load microfluidic droplets with two or more SPMs and one hydrogel bead (FIG. 11B). In a particular embodiment the flow rates of inlets were in the range of 100 ul/h for RT mix, 10-100 ul/h for SPMs, 150 ul/h for hydrogel beads and 800 ul/h for droplet stabilization oil. However, the flow rates of all phases can be adjusted independently between 1 and 10,000 ul/h, depending on the particular application.

The composition of the reverse transcription (RT) reaction mix is indicated in the Table 8 below.

TABLE 8
The composition of the RT reaction mix

| Reagent | Volume, μl |
|---|---|
| 5X RT buffer | 30 |
| dNTPs, 10mM each | 15 |
| TSO primer, 0.5 mM | 7.5 |
| (SEQ ID 5) | |
| 5′-AAG CAG TGG TAT | |
| CAA CGC AGA GTA CAT | |
| rGrGrG-3′ | |
| Igepal CA-630, 10% | 9 |
| Maxima H minus, 200 U/μl | 15 |
| Ribolock, 40 U/μl | 15 |
| Water | Up to 150 |

Droplets carrying co-encapsulated SPMs, hydrogel beads and assay reagents were collected off-chip and incubated at 42° C. for 90 min to perform a barcoding of the modified nucleic acids, followed by 15 min incubation at 85° C. To release the barcoded nucleic acid the droplets were broken by adding 20% perfluoro-octanol. To dissolve the SPMs the post-RT mix was supplemented with 0.5 μL of 20 mg/mL Proteinase K and incubate at 50° C. for 5 minutes. The aqueous phase was passed through Zymo Spin-IC column at 1000 g for 5 min. The flow-through fraction was collected diluted to 150 μl, purified 2-times with 0.8× volume of AMPure magnetic beads, and eluted in 21 μl of water.

The barcoded-cDNA was amplified by 19-cycles of PCR using Kapa HiFi Ready Mix and 0.5 μM of forward primer PCR1_p5_2020rz: 5′-TACGGCGACCACCGAGATC-3′ (SEQ ID 10) and 0.5 μM of reverse primer PCR1_tso_2020rz: 5′-AAGCAGTGGTATCAACGCAGAG-3′ (SEQ ID 11) following the thermocycling conditions indicated in Table 9 below.

TABLE 9
PCR cycling conditions

| Step | Temperature | Time |
|---|---|---|
| Initial denaturation | 98° C. | 00:03:00 |
| Denaturation | 98° C. | 00:00:15 |
| Annealing | 67° C. | 00:00:20 |
| Extension | 72° C. | 00:01:00 |

Go to step 2, 18 cycles (19 in total)

| | | |
|---|---|---|
| Final extension | 72° C. | 00:01:00 |
| Hold | 4° C. | Hold |

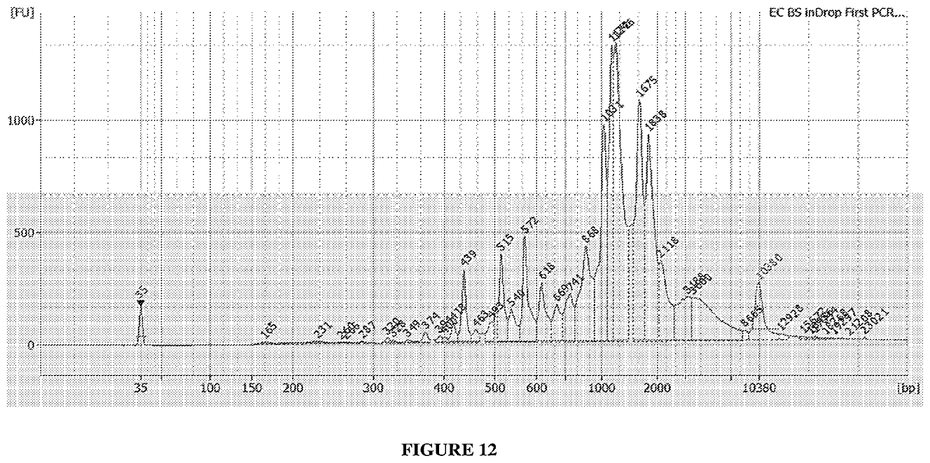

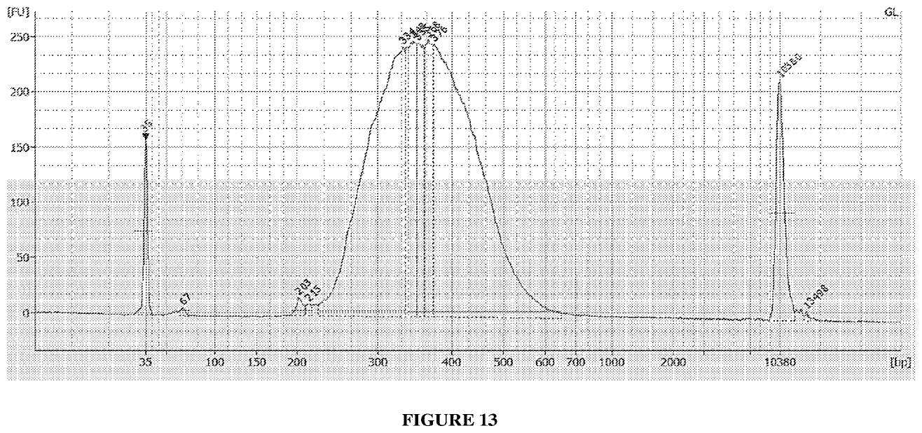

The amplified cDNA was twice purified with 0.6× volume AMPure magnetic beads and eluted into 20 μl of water. The resulting cDNA profile is indicated in FIG. 12. The cDNA was then fragmented by preparing the reaction mix indicated in Table 4. Samples were vortexed and spin-down and DNA fragmentation was carried out at 37° C. for 6 min, followed by reaction inactivation for 30 min at 65° C. The reaction was purified (double size selection) using 0.6× and 0.8× volume of SPRI beads. The purified fragmented DNA was eluted in 17.5 μL of water and ligated to adapter using following reaction mix (Table 5). Ligation was carried out at 20° C. for 15 min (lid at 30° C.). After reaction total volume was brought to 100 μL, 0.8× AMPure was performed and ligation product was eluted in 40 μL. Next indexing PCR was conducted by preparing the PCR reaction mix (Tables 6) and cycling conditions (Table 7). The amplified DNA library was purified using double size selection (0.6-0.8× SPRI beads) and eluted in 15 μL. The DNA library quality was then verified on Agilent BioAnalyzer HS DNA chip, as shown in FIG. 13. The DNA library was then sequenced using Illumina MiSeq 150 cycles kit.

### Example 6

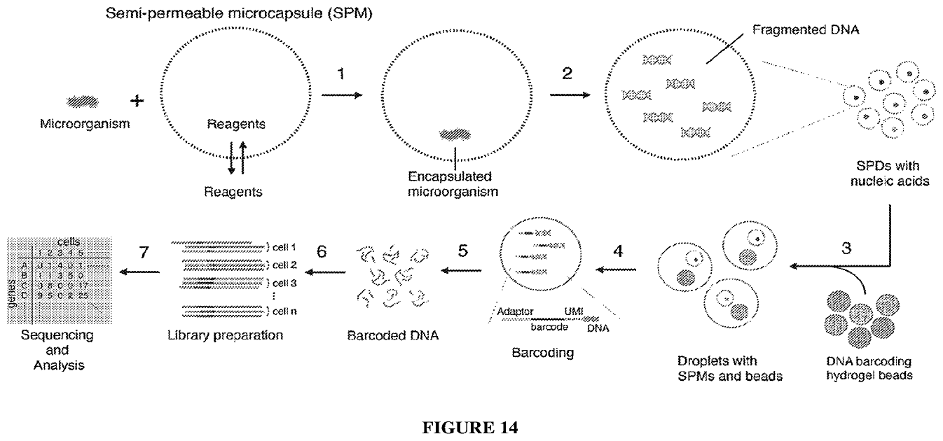

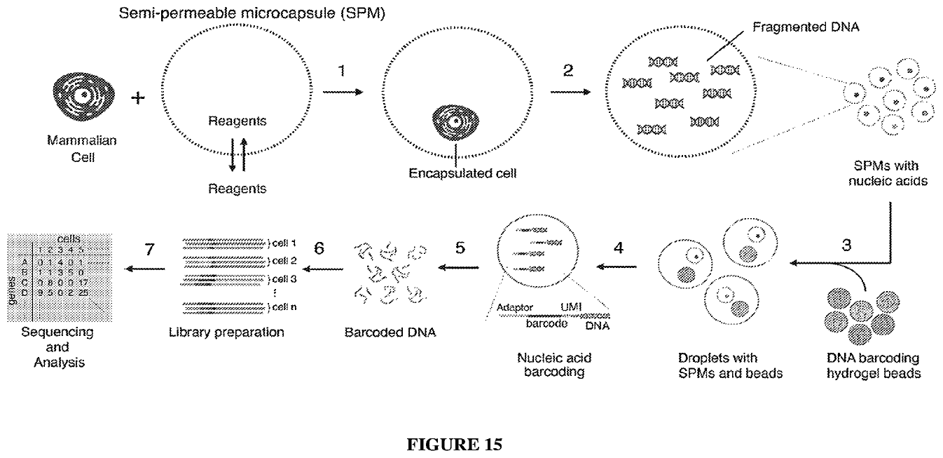

This example illustrates certain techniques for fragmenting the nucleic acids derived from single cells followed by the barcoding of said fragmented nucleic acids. The overall strategy of this particular example is best understood along with FIG. 14 and FIG. 15. In an exemplary embodiment, the individual biological species such as mammalian cells or bacteria are isolated in SPMs such that majority of SPMs contains one or no biological species. The compartmentalized cells are lysed to generate single-cell lysates retaining most of the nucleic acids inside the SPMs. The SPMs carrying single-cell lysates including nucleic acids such as genomic DNA are further processed to fragment the nucleic acid molecules. Various DNA fragmentation reagents are known to a person experienced in the field and some of these approaches/reagents, but not limited to, are listed below: transposase driven nucleic acid fragmentation, nuclease enzyme driven fragmentation, deoxyribonuclease driven fragmentation, endonuclease driven fragmentation, restriction endonucleases driven fragmentation, ultrasound driven fragmentation or using chemical complexes that generate hydroxyl radicals, such as iron-EDTA, can also be used to introduce random DNA cleavage. The SPMs having cell lysate including fragmented DNA are co-encapsulated along with hydrogel beads and assay reagents within microfluidic droplets. The hydrogel bead carries barcoding DNA oligonucleotides covalently attached to them that can be released by chemical, physical or enzymatic means. For example, once encapsulated the barcoding oligonucleotides may be released by melting the hydrogel bead in the presence of chemical agent (e.g. reducing agent DTT). Alternatively, the barcoding oligonucleotides may be released by photo-illumination or enzyme-driven hydrolysis. The nucleic acid molecules are attached to the barcoding oligonucleotide tags through the enzymatic (e.g. ligation, primer extension, PCR) or chemical (e.g. click-chemistry) reaction. It should be understood that certain applications may rely on nucleic molecule capture and barcoding without releasing barcoding oligonucleotides from the beads. For example, fragmented nucleic acid molecules of lysed cell could be captured on a bead within a droplet. The assay reagents introduced in microfluidic droplets may contain enzymes that can ligate DNA fragments to barcoding DNA oligonucleotides, extend barcoding DNA oligonucleotides at 3′ end or amplify the DNA fragments with DNA oligonucleotides.

The SPMs suspended in aqueous buffer can be packed (concentrated) such that their delivery into droplets becomes ordered and synchronized, enabling loading of a desirable number of SPMs into a droplet, such as exactly one SPM, two SPMs, three SPMs, etc. Likewise, the hydrogel beads carrying barcoding DNA primers suspended in aqueous buffer can also be packed (concentrated) such that their delivery into droplets becomes ordered and synchronized, ensuring that the majority of droplets host exactly one bead. As a result, the co-delivery of SPM(s) and bead(s) into the same droplet can be precisely controlled and high co-occupancy events (e.g., one SPM and one bead) can be achieved. In a typical scenario over 50% of droplets will contain one SPM and one bead. However, some assays and experimental conditions may prefer multiple (more than one) SPMs per droplet. For example, when majority of SPMs contain no cell it may be beneficial to load multiple SPMs into a droplet to increase the probability one of the encapsulated SPMs will carry a cell lysate including fragmented nucleic acids.

As one non-limiting example, the SPMs, hydrogel beads, and biological and/or chemical reagents are loaded in droplets using a microfluidics chip using the rates between 1 and 10,000 μl/h. Once encapsulated, the fragmented nuclei acid molecules inside the SPMs may be released by dissolving (breaking) the SPM shell using enzymatic or chemical means. For example, SPM shell can be disintegrated using collagenase enzyme. Alternatively, the fragmented nuclei acid molecules inside the SPMs can be processed further without breaking the SPM. Irrespectively whether the SPMs are disintegrated (broken) or not, the fragmented nucleic molecules can be tagged by oligonucleotides. It may be preferable to use barcoding DNA oligonucleotides to tag fragmented nucleic acid molecules. However, in some cases, it may be preferable to first attach adapters to the fragmented nucleic acids and only then barcode the resulting DNA fragments with barcoding DNA oligonucleotides. In a non-limiting example, fragmented nucleic acids, with or without adapters, can be ligated to barcoding DNA oligonucleotides. In another non-limiting examples, the fragmented nucleic acids, with or without adapters, can be tagged by primer extension reaction, DNA replication, or PCR.

The barcoded nucleic acid fragments may be released from droplets and/or SPMs can be broken by chemical or physical techniques. Typically, the emulsion droplets are broken by adding ≥10% perfluoro-octanol onto the collected emulsion. It should be understood that the decomposition (breakup) of SPMs will depend on their chemical composition and can rely on chemical (e.g. use of alkaline solution) or enzymatic (e.g. use of hydrolase, collagenase, protease) treatment.

The barcoded nucleic acid molecules released from droplets and/or SPMs can be amplified by PCR. The amplified DNA can then be further processed to construct sequencing library. Indexing PCR may be included to construct sequencing libraries and the primary sequence of nucleic acids, including the barcode and unique molecular identifiers can be determined by sequencing. It should be understood that the above example can be used to prepare and analyze, as non-limiting examples, genomes, transcriptomes, epigenomes, single nucleotide polymorphisms, specific gene expression levels, non-coding RNA, entire genes or their sections, etc.

### Example 7

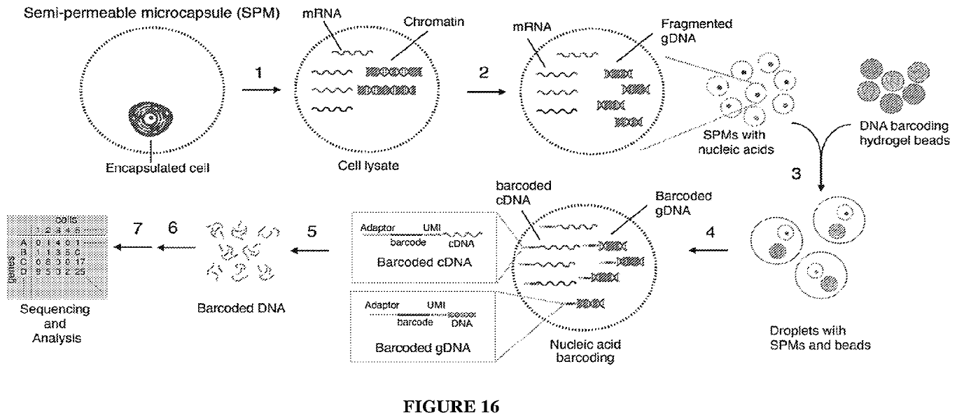

This example illustrates certain techniques for fragmenting the chromatin derived from single cells followed by the barcoding of chromatin fragments along with mRNA of the same cell. The overall strategy of this particular example is best understood along with FIG. 16. In an exemplary embodiment, the individual biological species such as cells are isolated in SPMs such that majority of SPMs contains one or no cell. The compartmentalized cells are lysed to generate single-cell lysates that retain most of the nucleic acids inside the SPMs. The SPMs carrying single-cell lysates including nucleic acids such as chromatin DNA and mRNA are further processed to fragment the chromatin DNA. Various DNA fragmentation reagents are known to a person experienced in the field and some of these approaches/reagents, but not limited to, are listed below: transposase driven nucleic acid fragmentation, nuclease enzyme driven fragmentation, deoxyribonuclease driven fragmentation, endonuclease driven fragmentation, restriction endonucleases driven fragmentation, ultrasound driven fragmentation or using chemical complexes that generate hydroxyl radicals, such as iron-EDTA, can also be used to introduce random DNA cleavage. The SPMs having cell lysate including fragmented chromatin DNA are co-encapsulated along with hydrogel beads and assay reagents within microfluidic droplets. The hydrogel bead carries barcoding DNA oligonucleotides covalently attached to them that can be released by chemical, physical or enzymatic means as detailed in above examples. It should be understood that certain applications may rely on fragmented chromatin and/or mRNA capture on the bead within a droplet. The assay reagents introduced in microfluidic droplets may contain enzymes that can ligate DNA fragments to barcoding DNA oligonucleotides, replicate nucleic acids, extend barcoding DNA oligonucleotides at 3′ end or amplify the DNA fragments with DNA oligonucleotides.

As mentioned above, the SPMs and hydrogel beads can be packed (concentrated) such that their delivery into droplets becomes ordered and synchronized, enabling loading of a desirable number of SPMs and hydrogel beads into a droplet. As a result, the co-delivery of SPM(s) and bead(s) into the same droplet can be precisely controlled and high co-occupancy events (e.g., one SPM and one bead, two SPMs and one bead, etc.) can be achieved.

As one non-limiting example, the SPMs, hydrogel beads, and biological and/or chemical reagents are loaded in droplets using a microfluidics chip (FIG. 6) using the rates between 1 and 10,000 μl/h. Once encapsulated, the fragmented nuclei acid molecules inside the SPMs may be released by dissolving (breaking) the SPM shell using enzymatic or chemical means. For example, SPM shell can be disintegrated using collagenase enzyme, dextranase enzyme, or others. Alternatively, the fragmented nuclei acid molecules inside the SPMs can be processed further without breaking the SPM. Irrespectively whether the SPMs are disintegrated (broken) or not, the fragmented nucleic molecules can be tagged by oligonucleotides. The said oligonucleotides may carry cell barcodes and UMIs as detailed above. In some cases, it may be preferable to first attach adapters to the fragmented nucleic acids and only then barcode the resulting DNA fragments with barcoding DNA oligonucleotides. In a non-limiting example, fragmented nucleic acids, with or without adapters, can be ligated to barcoding DNA oligonucleotides. In another non-limiting examples, the fragmented nucleic acids, with or without adapters, can be tagged by primer extension reaction, DNA replication, or PCR.

The mRNA molecules in the same droplet may be converted to cDNA using a reverse transcription (RT) reaction. During the RT step the barcoding nucleotides may anneal to poly(A) part of mRNA molecules via 3′ end poly(T) tail, and get extended to barcoded-cDNA. In another non-limiting example, mRNA molecules inside the SPMs can be ligated to barcoding DNA oligonucleotides. In yet another non-limiting example, it might be preferable to first synthesize cDNA molecules inside the SPMs and only then use nucleic acid barcoding reaction to tag cDNA with barcoding DNA oligonucleotides.

The barcoded nucleic acid fragments (e.g. cDNA and chromatin DNA) may be released from droplets and/or SPMs can be broken by chemical or physical techniques as described above and amplified by PCR. The amplified DNA can then be further processed to construct sequencing library. Indexing PCR may be included to construct sequencing libraries and the primary sequence of nucleic acids, including the barcode and unique molecular identifiers can be determined by sequencing.

### Example 8

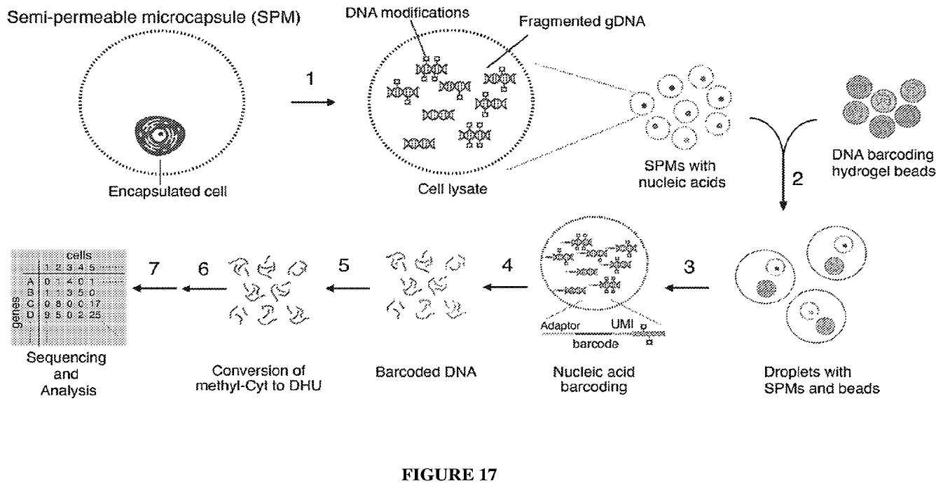

This example illustrates certain techniques for preparing single-cell methylome libraries. The overall strategy of this particular example is best understood along with FIG. 17. Similar to exemplary embodiments described above, the individual biological species such as cells are isolated in SPMs such that majority of SPMs contains one or no cell. The compartmentalized cells are lysed to generate single-cell lysates that retain most of the nucleic acids inside the SPMs. The SPMs carrying single-cell lysates including nucleic acids such as genomic DNA (gDNA) that may and may not be methylated, are further processed to fragment the said gDNA. Various DNA fragmentation reagents are known to a person experienced in the field are mentioned above. The SPMs having cell lysate including fragmented gDNA are co-encapsulated along with hydrogel beads and assay reagents within microfluidic droplets.

The hydrogel bead carries barcoding DNA oligonucleotides covalently attached to them that can be released by chemical, physical or enzymatic means as detailed in above examples. It should be understood that certain applications may rely on fragmented gDNA capture on the bead within a droplet. The assay reagents introduced in microfluidic droplets may contain enzymes that can ligate DNA fragments to barcoding DNA oligonucleotides, replicate nucleic acids, extend barcoding DNA oligonucleotides at 3′ end or amplify the DNA fragments with DNA oligonucleotides.

As mentioned above, the SPMs and hydrogel beads can be packed (concentrated) such that their delivery into droplets becomes ordered and synchronized, enabling loading of a desirable number of SPMs and hydrogel beads into a droplet. As a result, the co-delivery of SPM(s) and bead(s) into the same droplet can be precisely controlled and high co-occupancy events (e.g., one SPM and one bead, two SPMs and one bead, etc.) can be achieved.

As one non-limiting example, the SPMs, hydrogel beads, and biological and/or chemical reagents are loaded in droplets using a microfluidics chip (FIG. 6) using the rates between 1 and 10,000 ul/h. The fragmented gDNA may be tagged by oligonucleotides. The said oligonucleotides may carry cell barcodes and UMIs as detailed above. In some cases, it may be preferable to first attach adapters to the fragmented nucleic acids and only then barcode the resulting DNA fragments with barcoding DNA oligonucleotides. In a non-limiting example, fragmented nucleic acids, with or without adapters, can be ligated to barcoding DNA oligonucleotides. In another non-limiting examples, the fragmented nucleic acids, with or without adapters, can be tagged by primer extension reaction, DNA replication, or PCR.

The barcoded nucleic acid fragments may be released from droplets and/or SPMs can be broken by chemical or physical techniques as described above. The barcoded DNA molecules can be treated chemically or enzymatically treated to convert modified bases to another base analog. For example, the barcoded DNA fragments can then be treated to bisulfite conversion [36], a process in which the deamination of unmethylated cytosines into uracils occurs, while methylated cytosines (both 5-methylcytosine and 5-hydroxymethylcytosine) remain unchanged. Alternatively, the fragmented DNA can be subjected to TET/pyridine borane treatment [37]. Treated DNA fragments can be purified, converted to DNA library by adding sequencing adapters and sequenced. Indexing PCR may be included to construct sequencing libraries and the primary sequence of nucleic acids, including the barcode and unique molecular identifiers can be determined by sequencing.

### Example 9

This example illustrates the sequencing results of nucleic acids derived from single-cells encapsulated in microcapsules, using the method of this disclosure. First, a mixture comprising an even ratio of mouse NIH: 3T3 and human K562 cells were isolated in microcapsules as explained in Example 1. Next, the genomic DNA was depleted as explained in Example 2. Next, a plurality of microcapsules comprising nucleic acids was loaded into a plurality of microfluidic droplets along with hydrogel beads as explained in Example 3. In parallel, the scRNA-Seq library (without using the microcapsules) was prepared following the inDrops protocol [30]. The sequencing library prepared according to inDrops protocol contained approximately 4000 cells, and sequencing library prepared following Examples 1-3 contained in total approximately 4000 cells, whereas approximately 2000 cells were barcoded in the absence of collagenase A enzyme, and another 2000 cells were barcoded in the presence of collagenase A enzyme. The collagenase A was loaded in droplets along with RT reagents. The presence of collagenase A enzyme in microfluidic droplet disintegrated the microcapsules and as a result the encapsulated nucleic acids were released into a droplet milieu. Following cDNA synthesis using barcoded DNA oligonucleotides, the barcoded-cDNA was released from droplets, purified and prepared for sequencing as explained in Example 3.

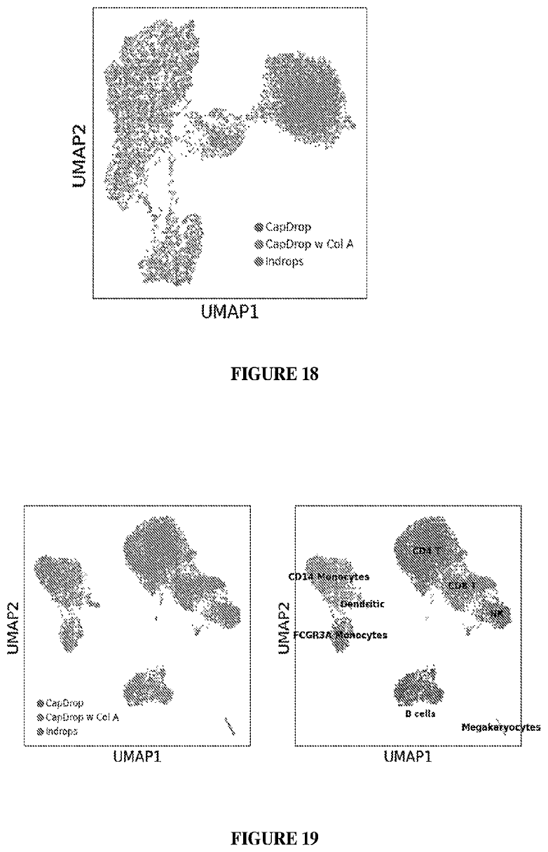

Sequencing of DNA libraries was performed on NextSeq Illumina instrument using NextSeq 500/550 High Output Kit v2.5 (75 Cycles) and following cycling numbers: Read 1-16 cycles, Read 2-62 cycles, i5 read-8 cycles, i7 read-6 cycles. The sequencing data was processed using STARsolo and the results obtained are presented in Table 10 below. According to the obtained metrics the single-cell transcriptomics libraries prepared following the method of disclosure (CapDrop) had higher percentage of reads mapped to genes and higher fraction of reads mapped to cells. At similar sequencing depth, CapDrop generated higher UMI count and gene counts than regular inDrops libraries. CapDrop also showed lower ribosomal gene counts (33.3%) as compared to standard inDrops (40%). As expected, CapDrop showed similar cell doublet ratio as inDrops-8.12% vs 6.52%. After batch correction using Harmony package, and projecting the single-cell transcriptomes on Uniform Manifold Approximation and Projection (UMAP), confirmed that there were no significant differences between CapDrop and inDrops (FIG. 18), suggesting that the method of this invention faithfully captures nucleic acids derived from single-cells without obvious biases. The results also show that adding collagenase A to reverse transcription reaction mix does not have a significant effect on UMI and gene capture.

TABLE 10
summarizing the sequencing results of scRNA-Seq libraries
prepared using CapDrop and inDrops methods.

| | | CapDrop with | |
|---|---|---|---|
| | CapDrop | collagenase | inDrops |
| Number of Reads | 58593990 | 68395042 | 80169415 |
| Reads With Valid Barcodes | 0.914 | 0.919 | 0.926 |
| Sequencing Saturation | 0.377 | 0.386 | 0.336 |
| Q30 Bases in CB + UMI | 0.948 | 0.949 | 0.953 |
| Q30 Bases in RNA read | 0.859 | 0.859 | 0.865 |
| Reads Mapped to Genome: Unique + Multiple | 0.929 | 0.931 | 0.905 |
| Reads Mapped to Genome: Unique | 0.617 | 0.619 | 0.565 |
| Reads Mapped to Gene: Unique + Multipe Gene | 0.747 | 0.752 | 0.658 |
| Reads Mapped to Gene: Unique Gene | 0.701 | 0.705 | 0.617 |
| Estimated Number of Cells | 2247 | 2664 | 4267 |
| Unique Reads in Cells Mapped to Gene | 39640849 | 45275829 | 44550737 |
| Fraction of Unique Reads in Cells | 0.965 | 0.938 | 0.901 |
| Mean Reads per Cell | 17641 | 16995 | 10440 |
| Median Reads per Cell | 16835 | 15926 | 9373 |
| UMIs in Cells | 24372617 | 27221325 | 29056576 |
| Mean UMI per Cell | 10846 | 10218 | 6809 |
| Median UMI per Cell | 10942 | 10298 | 6394 |
| Mean Gene per Cell | 2739 | 2665 | 1885 |
| Median Gene per Cell | 2772 | 2712 | 1893 |
| Total Gene Detected | 32042 | 32495 | 33967 |

In another experiment the CapDrop method was applied on human PBMC cells following the same procedure as described in Example 1-3. After batch correction using Harmony package, and projecting the single-cell transcriptomes on UMAP, once again confirmed that method of this invention faithfully identifies individual cell types based on their gene expression signatures (FIG. 19).

### Example 10

This example illustrates the sequencing results of nucleic acids derived from single bacteria cells encapsulated in microcapsules, using the method of this disclosure.

Bacteria cell preparation: 50 μL of night B. subtilis 23857 culture was inoculated in 5 mL pre-warmed LB Miller media and grow B. subtilis cells at 30° C. for 5.5 hours. Inoculate 5 μL of night E. coli MG1655 culture in 5 mL of fresh, pre-warmed LB Miller media and grow E. coli cells at 30° C. for 4.5 hours. 1 mL of each bacteria suspension was centrifuged at 1000 g for 5 minutes, washed twice with 1×DPBS containing 0.1% Pluronic F-68 and once in 1× DPBS. Next, cells were diluted in 1×DPBS up to OD600 value˜2.0.

Bactria cell encapsulation: To perform E. coli and B. subtilis co-encapsulation, 1.25 μL of cell suspension (OD˜ 2.0) was combined with 97.5 μL of 15% Dextran (MW 500k) and loaded onto microfluidics chip. Solution comprising 15% (w/v) dextran with cells and solution comprising 3% (w/v) were injected into a microfluidics chip (FIG. 3). Encapsulation was performed using 20 μm co-flow device and the following flow rates: 50 μL/h for GMA, 20 μL/h for dextran with cells and 200 μl/hr for carrier oil with surfactant. Encapsulation was performed for 2 hours and collected emulsion was incubated at 4° C. for 40 minutes. Approximately 500 μL of ice-cold 1×DPBS with 0.1% F-68 was added on top of emulsion and microcapsules were released by breaking the emulsion with 20% PFO. The microcapsule suspension was transferred to a new 1.5 mL tube, supplemented with 0.1% LAP and photo-polymerized under exposure to 405 nm light emitting diode (LED) device (Droplet Genomics, DG-BR-405) for 20 seconds. The resulting cross-linked microcapsules were washed twice in a capsule recovery buffer.

Cell recovery (optional): Resuspend microcapsules in 1-2 mL of LB media and transfer into a small Petri dish and incubate at 30° C. incubator for 30 minutes. After the incubation collect all capsules in 1.5 mL tube, spin down capsules at 1000 g for 2 minutes and proceed to bacteria lysis.

Bacteria lysis: Close-packed capsules were immersed in 1 mL Lysis Buffer 1 without lysozyme and after incubation for 5 min at room temperature spun down at 1000 g for 2 minutes. Supernatant was discarded. Next, the microcapsules were suspended in 1 mL of Lysis Buffer 1 supplemented with lysozyme and incubated at room temperature for 15 minutes. Next, microcapsules were centrifuged at 1000 g for 2 minutes, the supernatant was aspirated and microcapsules were resuspended in 1 mL of lysis buffer 2, and incubate at room temperature for 5 minutes. Next, microcapsules were centrifuged at 1000 g for 2 minutes, the supernatant was aspirated and microcapsules resuspended in the lysis buffer 2, incubated for a few minutes and spun down to remove the supernatant. Next, microcapsules were washed 4-5 times in capsule washing buffer and resuspended in 300 μL of washing buffer. Staining an aliquot of microcapsules with 1×SYBGR Green I indicated that ˜15% of microcapsules were fluorescent (contained bacterial cells). Finally, 300 μL of close-packed microcapsules were mixed with 700 μL of ice-cold ethanol (96%), transferred to −20° C. for storage.

DNA depletion: Microcapsules suspension in ethanol was let to equilibrate on ice for 5 minutes, washed in washing buffer 3-times and then treated with the DNAse I enzyme using reaction composition indicated in Table 11:

TABLE 11
DNA depletion reaction mix

| Volume, μL | Material | Final concentration | |
|---|---|---|---|
| 68 | Water, nuclease-free | — | |
| 20 | 10x DNaseI buffer + MgCl₂ | 1x | |
| 10 | 1 U/μL DNaseI | 0.05 | U/μL |
| 2 | 40 U/μL RiboLock RI | 0.4 | U/μL |

| | | |
|---|---|---|
| 100 | Close-packaged microcapsules | — |
| 200 | Final | — |

Reaction mix was incubated at 37° C. for 20 minutes and then additional 5 μL of DNaseI was added and reaction mix incubated further for 10 minutes. Microcapsules were washed 4-5 times with washing buffer and then proceeded to polyadenylation.

Polyadenylation: The reaction mix was prepared as indicated in Table 12 and incubated at 37° C. for 30 minutes. Next, microcapsules were washed 2-3 times in a capsule washing buffer and concentrated by centrifugation at 2000 g for 5 minutes, and proceeded to next step.

TABLE 12
Polyadenylation reaction mix

| Volume, μL | Material | Final | |
|---|---|---|---|
| 50 | Water, nuclease-free | — | |
| 20 | 10X E. coli Poly(A) Polymerase | 1x | |
| | Reaction Buffer | | |
| 20 | ATP (10 mM) | 1 | mM |
| 10 | E. coli Poly(A) Polymerase | 0.4 | U/μL |

| | | |
|---|---|---|
| 100 | Close-packed microcapsules | — |
| 200 | Final | — |

Nucleic acid barcoding: The DNA barcoding hydrogel beads and microcapsules (comprising polyadenylated RNA) were rinsed in a loading buffer (1× RT buffer, 0.6% Igepal CA-630) and loaded separately into a microfluidics chip (FIG. 5) along with RT reaction mix (Table 13).

TABLE 13
RT reaction mix composition

| Volume, μL | Material | Final (in droplets) | |
|---|---|---|---|
| 30 | 5X RT buffer | 1X | |
| 11.25 | dNTPs, 10 mM each | 0.5 | mM |
| 4.5 | TSO, 50 μM | 1 | uM |
| 6.75 | Igepal CA-630, 10% | 0.3% | |
| 11.25 | Maxima H minus RT, 200 U/μL | 10 | U/μL |
| 11.25 | RiboLock, 40 U/μL | 3 | U/μL |

| | | |
|---|---|---|
| 75 | Water, nuclease-free | — |
| 150 | Final | — |

The infusion flow rates used were: 200 μL/hr-RT mix, 40-80 μL/hr-hydrogel beads, 10-40 μL/hr-microcapsules, 500 μL/hr-carrier oil. The emulsion was collected in 1.5 mL tube prefilled with 200 μL of light mineral oil, on ice.

Reverse transcription: The barcoding DNA primers were released from the hydrogel beads by exposing emulsion droplets under UV lamp for 7 minutes [38]. The emulsion was transferred to 42° C. for 60 minutes, followed by 85° C. for 5 minutes. After RT step, the emulsion was broken by adding 10 μL of PFO, diluted to 100 μL with 1× RT buffer and digested with 1 μL of dextranase (Sigma, D0443-50ML) at 37° C. for 5 minutes. To dissolve microcapsule's shell the post-RT mix was treated with 1 μL of 20 mg/mL Proteinase K and incubate at 37° C. for 10 minutes. Hydrogel beads were separated from barcoded cDNA with Zymo Spin-IC column, by centrifugation at 1000 g or 5 min.

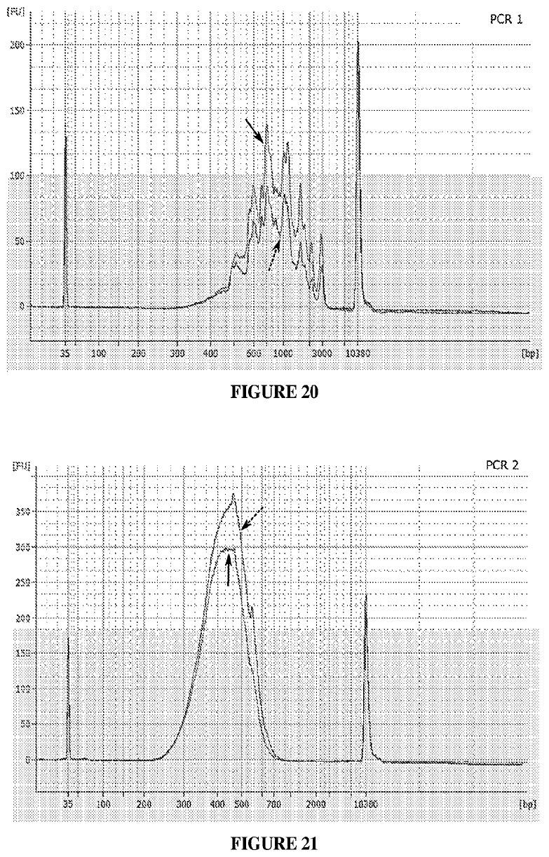

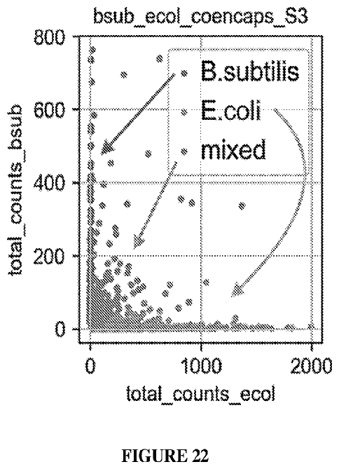

Barcode cDNA purification and amplification: 0.8× AMPure purification was performed twice and eluted in 21 μL of water and prepared for sequencing as detailed in Example 3 above. The amplified cDNA library is presented in FIG. 20 and final library ready for sequencing is presented in FIG. 21. The Sequencing of DNA libraries was performed on MiSeq Illumina instrument using following cycling numbers: Read 1-16 cycles, Read 2-134 cycles, i5 read-8 cycles, i7 read-6 cycles. Sequencing data was processed using STARsolo. The obtained reads were demultiplexed according to the cell barcodes, the reads aligned to E. coli and B. subtilis bacterial genomes and the transcript count of one species (e.g., E. coli) was plotted as a function of transcript count of another species (e.g., B. subtilis). The results presented in FIG. 22 show that using the method of this disclosure transcriptomics of thousands of individual bacterium cells can be quantified with high precision and at increased throughput.

### OTHER EXAMPLES

This example illustrates various methods and systems used in the above examples.

Microfluidic device design. The design of the microfluidics devices used in some of these examples is indicated in FIG. 3 and FIG. 6. The design of semi-permeable microcapsule (SPM) generation device is indicated in FIG. 3. The device contains three inlets for i) aqueous phase rich in shell-forming compound; ii) for aqueous phase that contains dispersed cells and is rich in core-forming compound; iii) the carrier oil. Device contains one outlet for droplet collection off-chip. The device includes flow focusing junction where aqueous and oil phases meet, and biological sample encapsulation occurs.

The design of nucleic acid barcoding device is indicated in FIG. 6 and contains four inlets for, i) hydrogel beads carrying barcoding DNA oligonucleotides, ii) SPMs, iii) assay reagents (e.g., nucleic acid modification/amplification reaction mix), iv) carrier oil with surfactant. The device also contains one outlet port for droplet collection. The device includes two junctions, one for bringing the three aqueous inputs together, and a second junction for sample encapsulation, where aqueous and oil phases meet and droplet generation occurs. To stabilize drops against coalescence droplet stabilization oil (Droplet Genomics) was used.

Soft lithography. The microfluidic device with rectangular microfluidic channels 80 micrometers deep was manufactured following established protocol [39].

Microfluidic device operation. As stated above, throughout the experiments the flow rates for introducing fluids into microfluidics device may be tuned in the range of 10-5000 μl/h. Each aqueous phase was injected into the microfluidic device via polyethylene tubing (ID 0.38×OD 1.09 mm, BB31695-PE/2) connected to a needle of a sterile 1 ml syringe (Braun) placed on a syringe pump (Harvard Apparatus, PC2 70-2226).

Cell cultivation. The K-562 and NIH/3T3 cells cell lines were maintained in DMEM supplemented with 10% (v/v) fetal bovine serum and 1% (v/v) penicillin-streptomycin at 37° C. in 5% CO₂ and 60-80% humidity atmosphere, at density˜3×10⁵ cells ml-1.

Single-cell isolation in SPMs. The cell encapsulation is a random process that can be characterized by Poisson statistics as described previously [39]. To minimize two or more cells from entering the same drop, diluted cell suspensions were used (˜100,000 cells/mL) to obtain an average occupancy of 1 cell in 5 droplets. In some specific embodiments, the cells were resuspended in 15% dextran (MW 500k) and then encapsulated using a microfluidics chip.

Loading hydrogel beads (HB) into the microfluidic device has been described previously [13, 30]. Prior to loading the HB were resuspended in a desirable assay buffer (e.g., 1× Maxima RT buffer, EP0742, Thermo Fisher Scientific) supplemented with 0.1-0.6% (v/v) IGEPAL CA-630. The close packed HB were injected into the microfluidic device through a tubing connected to a syringe placed on a syringe pump.

Loading SPMs into the microfluidic device. In a specific example, the SPMs carrying lysed cells were resuspended in 10 mM Tris-HCl [pH 7.5], 0.1% (v/v) Triton X-100 and injected into the microfluidic device through a tubing connected to a syringe placed on a syringe pump. It should be understood that other buffer may be used to resuspend and inject SPMs into a microfluidics device.

Barcoding inside droplets. After SPM, hydrogel bead and assay reagent co-encapsulation the barcoding DNA oligonucleotides were released from the HB by exposing the droplets to 405 nm light emitting diode (Droplet Genomics, DG-BR-405) for 20 seconds. Next, the droplets may be incubated at a desirable temperature for a desirable period of time to initiate a chemical or enzymatic reaction. For example, in a specific embodiment the droplets were heated to 50° C. and incubated for 1 hours to allow cDNA synthesis to occur, and then heated for 15 min at 70° C. to terminate the reaction. The emulsion was then broken (demulsified) by adding 10% emulsion breaker (Droplet Genomics, DG-EB-1). The aqueous phase from the broken droplets was transferred into a separate DNA LoBind tube (Eppendorf) and processed as described above.

All publications, patents, and patent applications mentioned herein are hereby incorporated by reference in their entirety. In cases where the present specification and a document incorporated by reference include conflicting and/or inconsistent disclosure, the present specification shall control.

### REFERENCES

1. Trapnell, C., Defining cell types and states with single-cell genomics. Genome Res, 2015. 25 (10): p. 1491-8.
2. Dalerba, P., et al., Single-cell dissection of transcriptional heterogeneity in human colon tumors. Nat Biotechnol, 2011. 29 (12): p. 1120-7.
3. Islam, S., et al., Characterization of the single-cell transcriptional landscape by highly multiplex RNA-seq. Genome Res, 2011. 21 (7): p. 1160-7.
4. Deng, Q., et al., Single-cell RNA-seq reveals dynamic, random monoallelic gene expression in mammalian cells. Science, 2014. 343 (6167): p. 193-6.
5. Shalek, A. K., et al., Single-cell RNA-seq reveals dynamic paracrine control of cellular variation. Nature, 2014. 510 (7505): p. 363-9.
6. Treutlein, B., et al., Reconstructing lineage hierarchies of the distal lung epithelium using single-cell RNA-seq. Nature, 2014. 509 (7500): p. 371-+.
7. Kumar, R. M., et al., Deconstructing transcriptional heterogeneity in pluripotent stem cells. Nature, 2014. 516 (7529): p. 56-U112.
8. Nagano, T., et al., Single-cell Hi-C reveals cell-to-cell variability in chromosome structure. Nature, 2013. 502 (7469): p. 59-64.
9. Smallwood, S. A., et al., Single-cell genome-wide bisulfite sequencing for assessing epigenetic heterogeneity. Nat Methods, 2014. 11 (8): p. 817-20.
10. Stergachis, A. B., et al., Developmental Fate and Cellular Maturity Encoded in Human Regulatory DNA Landscapes. Cell, 2013. 154 (4): p. 888-903.
11. Sigal, A., et al., Variability and memory of protein levels in human cells. Nature, 2006. 444 (7119): p. 643-6.
12. Buenrostro, J. D., et al., Single-cell chromatin accessibility reveals principles of regulatory variation. Nature, 2015. 523 (7561): p. 486-90.
13. Klein, A. M., et al., Droplet barcoding for single-cell transcriptomics applied to embryonic stem cells. Cell, 2015. 161 (5): p. 1187-201.
14. Macosko, E. Z., et al., Highly Parallel Genome-wide Expression Profiling of Individual Cells Using Nanoliter Droplets. Cell, 2015. 161 (5): p. 1202-14.
15. Zheng, G. X., et al., Massively parallel digital transcriptional profiling of single cells. Nat Commun, 2017. 8: p. 14049.
16. Gierahn, T. M., et al., Seq-Well: portable, low-cost RNA sequencing of single cells at high throughput. Nat Methods, 2017. 14 (4): p. 395-398.
17. Rosenberg, A. B., et al., Single-cell profiling of the developing mouse brain and spinal cord with split-pool barcoding. Science, 2018. 360 (6385): p. 176-182.
18. Mazutis, L., J. C. Baret, and A. D. Griffiths, A fast and efficient microfluidic system for highly selective one-to-one droplet fusion. Lab on a Chip, 2009. 9 (18): p. 2665-2672.
19. Mazutis, L. and A. D. Griffiths, Selective droplet coalescence using microfluidic systems. Lab on a Chip, 2012. 12 (10): p. 1800-1806.
20. Ahn, K., et al., Electrocoalescence of drops synchronized by size-dependent flow in microfluidic channels. Applied Physics Letters, 2006. 88 (26): p. 264105-3.
21. Chabert, M., K. D. Dorfman, and J. L. Viovy, Droplet fusion by alternating current (AC) field electrocoalescence in microchannels. Electrophoresis, 2005. 26 (19): p. 3706-3715.
22. Priest, C., S. Herminghaus, and R. Seemann, Controlled electrocoalescence in microfluidics: Targeting a single lamella. Applied Physics Letters, 2006. 89 (13): p. 134101.
23. Abate, A. R., et al., High-throughput injection with microfluidics using picoinjectors. Proc Natl Acad Sci USA, 2010. 107 (45): p. 19163-6.
24. Peterson, V. M., et al., Multiplexed quantification of proteins and transcripts in single cells. Nat Biotechnol, 2017. 35 (10): p. 936-939.
25. Stoeckius, M., et al., Simultaneous epitope and transcriptome measurement in single cells. Nature Methods, 2017. 14 (9): p. 865-+.
26. Lareau, C. A., et al., Droplet-based combinatorial indexing for massive-scale single-cell chromatin accessibility. Nat Biotechnol, 2019. 37 (8): p. 916-924.
27. Koster, S., et al., Drop-based microfluidic devices for encapsulation of single cells. Lab on a Chip, 2008. 8 (7): p. 1110-1115.
28. Clausell-Tormos, J., et al., Droplet-based microfluidic platforms for the encapsulation and screening of mammalian cells and multicellular organisms. Chem Biol, 2008. 15 (5): p. 427-137.
29. Abate, A. R., et al., Beating Poisson encapsulation statistics using close-packed ordering. Lab Chip, 2009. 9 (18): p. 2628-31.
30. Zilionis, R., et al., Single-cell barcoding and sequencing using droplet microfluidics. Nat Protoc, 2017. 12 (1): p. 44-73.
31. Christopher, G. F. and S. L. Anna, Microfluidic methods for generating continuous droplet streams. Journal of Physics D-Applied Physics, 2007. 40 (19): p. R319-R336.
32. Xia, Y. N. and G. M. Whitesides, Soft lithography. Angew Chem Int Ed, 1998. 37 (5): p. 551-575.
33. Brangwynne, C. P., P. Tompa, and R. V. Pappu, Polymer physics of intracellular phase transitions. Nature Physics, 2015. 11 (11): p. 899-904.
34. Mace, C. R., et al., Aqueous multiphase systems of polymers and surfactants provide self-assembling step-gradients in density. J Am Chem Soc, 2012. 134 (22): p. 9094-7.
35. Macosko, E. Z., et al., Highly Parallel Genome-wide Expression Profiling of Individual Cells Using Nanoliter Droplets. Cell, 2015. 161 (5): p. 1202-1214.
36. Frommer, M., et al., A Genomic Sequencing Protocol That Yields a Positive Display of 5-Methylcytosine Residues in Individual DNA Strands. Proceedings of the National Academy of Sciences of the United States of America, 1992. 89 (5): p. 1827-1831.
37. Liu, Y. B., et al., Bisulfite-free direct detection of 5-methylcytosine and 5-hydroxymethylcytosine at base resolution. Nature Biotechnology, 2019. 37 (4): p. 424-+.
38. Zilionis, R., et al., Single-cell barcoding and sequencing using droplet microfluidics. Nature Protocols, 2017. 12 (1).
39. Mazutis, L., et al., Single-cell analysis and sorting using droplet-based microfluidics. Nat Protoc, 2013. 8 (5): p. 870-91.

1.-50. (canceled)

51. A method comprising:
co-encapsulating a plurality of microcapsules and a plurality of particles in a plurality of droplets, wherein each particle of the plurality of particles comprises a molecular tag, and each microcapsule of the plurality of microcapsules comprises a core, a semi-permeable shell, and nucleic acid(s) obtained from a biological species, wherein the nucleic acid is comprised in the core, and wherein:
(i) the nucleic acid(s) has been obtained from the biological species inside the plurality of microcapsules prior to co-encapsulation,
(ii) the plurality of droplets comprises, on average, one of the plurality of microcapsules and one of the plurality of particles,
(iii) the molecular tag of the particle in one droplet is distinguishable from the molecular tags of the particles in the other droplets;
wherein the method optionally comprises releasing the molecular tag from the particle and/or releasing the nucleic acid from the microcapsule; and
attaching the molecular tag to the nucleic acid in the droplet.

52. The method of claim 51, wherein the molecular tag is a barcoding oligonucleotide, which allows the nucleic acid in one droplet to be uniquely identified from among the nucleic acids of the other droplets and is 12 to 300 nucleotides in length, preferably 20 to 150 nucleotides in length, and more preferably 30 to 120 nucleotides in length.

53. The method of claim 52, wherein the barcoding DNA oligonucleotide comprises one or more of
(i) a unique molecular identifier (UMI), preferably wherein the UMI is a random nucleotide sequence 4 to 50 nucleotides in length, and more preferably in the range of 4-16 nucleotides in length;
(ii) a cell barcode sequence preferably longer than 4 but shorter than 100 nucleotides, and preferably 6 to 70 nucleotides in length;
(iii) a sequence able to specifically bind to a region of interest in the nucleic acid such as a poly-(dT) sequence, gene specific sequence or a specific DNA sequence; and
optionally comprising
(iv) a sticky end, or a blunt end; and/or
(v) an adapter sequence that is complimentary to a target sequence such as PCR adapter, sequencing adapter, or ligation adapter.

54. The method of claim 53, wherein at least a portion of the barcoding DNA oligonucleotide binds to nucleic acid of interest; or wherein the barcoding DNA oligonucleotide gets attached to the nucleic acid of interest, or wherein the barcoding DNA oligonucleotide gets extended as a complimentary strand of nucleic acid of interest.

55. The method of claim 54, wherein attaching the barcoding DNA oligonucleotide to nucleic acid of interest may be conducted by ligase-driven reaction or click-chemistry reaction, and/or wherein attaching the barcoding DNA oligonucleotide by extension as a complimentary strand of nucleic acid of interest may be achieved by reverse transcriptase, DNA polymerase or RNA polymerase.

56. The method of claim 55, wherein the oligonucleotide attached to the nucleic acids may comprise molecular tag, molecular barcode, cell barcode, UMI, random sequence, sequencing adapter, PCR adapter, ligation adapter, RNA polymerase promoter, or any other nucleotide sequence that would facilitate the subsequent analysis of the said nucleic acid of interest.

57. The method according to claim 51, comprising attaching the molecular tags to the nucleic acid(s) of the microcapsules, to produce barcoded nucleic acid(s).

58. The method according to claim 51, breaking the shell of the co-encapsulated microcapsules in order to release the nucleic acid(s) and comprising attaching the molecular tags to the nucleic acid(s), to produce barcoded nucleic acid(s).

59. The method according to claim 51, comprising amplifying the barcoded nucleic acids to produce amplified nucleic acids.

60. The method of claim 51 comprising breaking the shell of the co-encapsulated microcapsules in order to release the nucleic acid(s) and attaching the molecular tags to the released nucleic acid(s) in the droplets, to produce barcoded nucleic acid(s),
wherein attaching the molecular tag to the nucleic acids comprises reverse transcription reaction, primer extension reaction, DNA replication, ligation reaction, or click-chemistry reaction, and
comprising breaking the droplets to release the barcoded nucleic acids and purifying the barcoded nucleic acids, and
comprising amplifying the barcoded nucleic acids to produce amplified nucleic acids.

61. The method of claim 51, comprising attaching the molecular tags to the nucleic acid(s) of the microcapsules, whereas microcapsules are in the droplets, to produce barcoded nucleic acid(s),
wherein attaching the molecular tag to the nucleic acids comprises reverse transcription reaction, primer extension reaction, DNA replication, ligation reaction, or click-chemistry reaction, and
comprising breaking the droplets to release the barcoded nucleic acids and purifying the barcoded nucleic acids, and
comprising amplifying the barcoded nucleic acids to produce amplified nucleic acids.

62. The method of claim 51, wherein the molecular tag is covalently attached to the particle and at least one of the molecular tags comprises a cleavable linker selected from one of photocleavable linker, chemically cleavable linker, or enzymatically cleavable linker.

63. The method of claim 51, wherein the biological species is a cell, a microorganism, a bacterium, a virus or a biological sample comprising nucleic acid, optionally wherein the biological sample is a cell-free sample.

64. The method of claim 51, further comprising, generating the plurality of microcapsules, wherein at least the majority of the generated plurality of microcapsules comprises no more than one cell.

65. The method of claim 51, further comprising, prior to the co-encapsulating, lysing the plurality of cells in plurality of microcapsules to obtain the plurality of microcapsules with cell lysates and treating the semi-permeable microcapsules comprising the lysates in order to deplete one or more enzymatic inhibitors present in the lysate, and optionally: (i) converting RNA in the cell lysate to cDNA in the microcapsules using a reverse transcriptase reaction; (ii) amplifying the nucleic acids in the microcapsules using a DNA polymerase driven reaction; (iii) fragmenting the nucleic acids in the microcapsules using chemical, enzymatic or physical means, and/or (iv) attaching the oligonucleotides to the fragmented nucleic acids.

66. The method of claim 51, wherein at least a majority of the plurality of droplets comprises on average one microcapsule per droplet and no more than one particle per droplet on average.

67. The method of claim 51, wherein the plurality of droplets is a plurality of water-in-oil droplets generated using a microfluidic device.

68. The method of claim 51, wherein each particle of the plurality of particles comprises a molecular tag with a unique cell barcode, and wherein the nucleotide sequence of the said cell barcode of one particle is different from the cell barcode sequence of other particles.

69. The method of claim 51, wherein the plurality of microcapsules have an average diameter of at least about 1 μm, preferably between 10-500 μm, and more preferably between 20-100 μm.

70. The method of claim 65, wherein the plurality of droplets have a volume of less than about 100 nanoliters, preferably a volume of about 0.1-10 nanoliters, and more preferably a volume of about 0.1 to 3 nanoliters.

71. The method of claim 51, wherein the plurality of particles is a plurality of hydrogel beads, preferably wherein the hydrogel beads have a size in the range of 1-200 μm and more preferably in the range of 10-80 μm, and even more preferably in the range of 20-70 μm.

72. A method for barcoding nucleic acid, comprising:
a) encapsulating a plurality of cells in a plurality of semi-permeable microcapsules such that the majority of the semi-permeable microcapsules contain no more than one cell;
b) lysing the encapsulated cells in order to produce a cell lysate comprising nucleic acids within the plurality of semi-permeable microcapsules;
c) treating the plurality of semi-permeable microcapsules comprising the lysates in order to purify the released nucleic acids and/or deplete one or more enzymatic inhibitors present in the lysates;
d) producing a plurality of microfluidic droplets using a microfluidic device, the plurality of microfluidic droplets comprising the plurality of semi-permeable microcapsules, a plurality of particles comprising molecular tags, and one or more assay reagents, wherein at least 1%, preferably at least 10%, more preferably at least 50%, of said plurality of droplets comprises a single microcapsule and a single particle, and one or more assay reagents;
e) labelling the nucleic acids with the molecular tags in the plurality of droplets to produce barcoded nucleic acids, where the nucleic acids from each cell comprise the same cell barcode which is different from the cell barcode of nucleic acids from other cells in other droplets; and
f) releasing the barcoded nucleic acids by breaking the plurality of droplets.

73. The method of claim 70, further comprising amplifying the nucleic acids using a DNA polymerase driven reaction following step c) and before step d), or further comprising amplifying the barcoded nucleic acids after e) and prior f) by DNA polymerase.

74. The method of claim 70, further comprising converting the released RNA to cDNA in the semi-permeable microcapsule using a reverse transcription reaction following step c) and before step d).

75. The method of claim 70, further comprising fragmenting the nucleic acids in the microcapsules using chemical, enzymatic or physical means following step c) and before step d).

76. A kit for attaching a molecular tag to a nucleic acid within in a water-in-oil droplet, the kit comprising:
(1) one or more particles comprising a molecular tag, wherein the molecular tag is a barcoding DNA oligonucleotide, and wherein preferably the molecular tag of one particle is different from the molecular tags of other particles;
(2) one or more reagents in aqueous solution suitable for creating the water-in-oil droplet;
(3) comprising a carrier oil supplemented with a surfactant, wherein the surfactant is suitable to stabilize the water-in-oil droplets and prevent against coalescence, and
(4) either: (i) one or more microcapsules, each microcapsule having a core, a semi-permeable shell and a biological sample comprising a nucleic acid comprised in the core; or
(ii) a shell precursor and a core precursor, suitable for making a semi-permeable microcapsule by aqueous phase separation in a further water-in-oil droplet.
(5) a microfluidic chip configured to produce the water-in-oil droplet, optionally comprising a second microfluidic chip configured to produce the further water-in-oil droplet from which the microcapsule is produced.


SEQUENCE LISTING (11 sequences)

SEQ ID NO: 1 - protein, 19 aa, unidentified
TACGGCGACCACCGAGATC

SEQ ID NO: 2 - protein, 22 aa, unidentified
AAGCAGTGGTATCAACGCAGAG

SEQ ID NO: 3 - protein, 33 aa, unidentified
GATCGGAAGAGCACACGTCTGAACTCCAGTCAC

SEQ ID NO: 4 - protein, 13 aa, unidentified
GCTCTTCCGATCT

SEQ ID NO: 5 - protein, 33 aa, unidentified
AAGCAGTGGTATCAACGCAGAGTACATRGRGRG

SEQ ID NO: 6 - protein, 29 aa, unidentified
AATGATACGGCGACCACCGAGATCTACAC

SEQ ID NO: 7 - protein, 50 aa, unidentified
CAAGCAGAAGACGGCATACGAGATCAGTGGGTGACTGGAGTTCAGACGTG

SEQ ID NO: 8 - protein, 50 aa, unidentified
CAAGCAGAAGACGGCATACGAGATGTTGTCGTGACTGGAGTTCAGACGTG

SEQ ID NO: 9 - protein, 50 aa, unidentified
CAAGCAGAAGACGGCATACGAGATTGACAAGTGACTGGAGTTCAGACGTG

SEQ ID NO: 10 - protein, 19 aa, unidentified
TACGGCGACCACCGAGATC

SEQ ID NO: 11 - protein, 22 aa, unidentified
AAGCAGTGGTATCAACGCAGAG